(19) United States
(12) Patent Application Publication
(10) Pub. No.: US 2014/0323321 A1
(43) Pub. Date: Oct. 30, 2014

(54) DETECTION OF HEAD AND NECK CANCER USING HYPERMETHYLATED GENE DETECTION

(71) Applicant: The Johns Hopkins University, Baltimore, MD (US)

(72) Inventor: Joseph A. Califano, Owings Mills, MD (US)

(73) Assignee: The Johns Hopkins University, Baltimore, MD (US)

(21) Appl. No.: 14/223,914

(22) Filed: Mar. 24, 2014

Related U.S. Application Data
(63) Continuation of application No. 12/933,996, filed on Dec. 13, 2010, now abandoned.

(51) Int. Cl. C12Q 1/68

(52) U.S. Cl. CPC C12Q 1/6886; C12Q 2600/154; C12Q 2600/118; C12Q 2600/106
USPC 506/4; 506/16

(57) ABSTRACT

Methods and kits for detection of a cell proliferative disorder, such as head and neck cancer are provided utilizing analysis of the methylation state of targeted genes or regulatory regions of genes in a saliva or serum sample are described. The presence of hypermethylation of the genes or their regulatory regions is indicative of the presence, or a stronger possibility of recurrence and or a poorer prognosis in subjects with cancer.

### CROSS REFERENCE TO RELATED APPLICATION(S)

This application is a continuation of U.S. application Ser. No. 12/933,996, filed Dec. 13, 2010, which is a 35 USC §371 National Stage application of International Application No. PCT/US2008/088341 filed Dec. 24, 2008, now expired; which claims the benefit under 35 USC §119(e) to U.S. Application Ser. No. 61/072,079 filed Mar. 27, 2008, now expired. The disclosure of each of the prior applications is considered part of and is incorporated by reference in the disclosure of this application.

### BACKGROUND OF THE INVENTION

1. Field of the Invention

The invention relates generally to methods and kits useful for detecting, diagnosing or evaluating cancer and more specifically to methods and kits for detecting, diagnosing or evaluating head and neck squamous cell carcinoma (HNSCC) by detecting methylation changes in the saliva and serum of subjects with a profile of gene markers.

2. Background Information

There are >40,000 new cases of head and neck squamous cell carcinoma (HNSCC) in the United States each year, with a mortality rate of 12,000 U.S. deaths annually. These incidence and mortality figures correspond to >4% of all new cancer cases and 2% of all cancer deaths in the United States each year. There have been modest improvements in survival for patients with HNSCC in the past 30 years. From 1995 to 2001, only ˜30% of the HNSCC in the Unites States have been diagnosed at an early clinical stage. Intuitively, early detection of HNSCC would improve clinical outcomes. Currently, there is no definitive evidence that widespread population screening using routine head and neck examination with or without fiberoptic endoscopy and/or vital staining would result in a decrease in mortality from HNSCC. However, there is evidence that screening high-risk populations may be cost-effective.

The use of molecular markers in body fluids for cancer detection has been explored with the intent to improve screening accuracy and cost effectiveness. Body fluids can potentially carry whole cells, as well as protein, DNA, and RNA species that allow for detection of cellular alterations related to cancer. Examples of relevant body fluids used for detection include: analysis of sputum for lung cancer diagnosis; urine for urologic tumors; saliva for HNSCC; breast fluid; as well as serum or plasma for almost all types of cancer. The feasibility of cancer detection in body fluids also opens a new potential for surveillance after treatment. Molecular detection could be useful to predict tumor recurrence before clinical symptoms or appearance of lesions having the potential to change treatment and follow-up approach.

An epigenetic pathway of transcriptional inactivation for many tumor suppressor genes includes CpG island hypermethylation within promoter regions. This pathway has been identified in many different cancers and recent studies have focused on promoter hypermethylation in HNSCC. Promoter hypermethylation in tissue samples can be detected by using quantitative methylation-specific PCR (Q-MSP); this real-time PCR methodology allows a more objective, robust, and rapid assessment of promoter methylation status. The ability to quantify the methylation provides the potential for determination of a threshold value of methylation to improve sensitivity and specificity in detection of tumor-specific signal.

The detection of DNA methylation in body fluids also has the potential to distinguish high-risk subjects that either harbor occult cancers or have a higher risk for development of cancer. Palmisano et al were able to detect aberrant promoter methylation in sputum of patients with squamous cell lung carcinoma up to 3 years before clinical diagnosis. (Palmisano et al., Cancer Res 2000; 60:5954-8) Subsequently, using a panel of genes, they were able to identify patients at high risk for cancer incidence by detecting DNA hypermethylation in sputum in a prospective study. Identification of differential promoter hypermethylation patterns between primary tumors and saliva or serum obtained from patients with HNSCC has already been shown in limited cohorts with a limited number of genes.

Unfortunately, the detection, evaluation and or prognosis of HNSCC using an expanded number of specifically targeted genes or regulatory regions of genes have not yet been described.

Furthermore, head and neck cancer is a disease that is often detected late, so that it requires morbid treatment or results in death.

### SUMMARY OF THE INVENTION

The present invention is based on the discovery of a panel of markers that provide an improved ability to detect epigenetic changes associated with HNSCC in salivary rinses and serum from patients with HNSCC. Further this panel of promoter hypermethylation markers can be used to anticipate the diagnosis of tumor recurrence by detecting the epigenetic changes associated with HNSCC.

The present invention relates to methods and kits used for diagnosing, or evaluating a subject having or at risk of developing head and neck cancer by determining the methylation state of a gene or the regulatory region of at least one gene in a nucleic acid sample from the subject, and wherein at least one gene or regulatory region is hypermethylated as compared to the same region in a corresponding normal cell.

In one embodiment, the invention provides a method for diagnosing a subject having or at risk of developing head and neck cancer. The method includes determining the methylation state of a gene or the regulatory region of at least two genes in a nucleic acid sample from the subject, wherein the at least two genes or regulatory regions are hypermethylated as compared to the same regions in a corresponding normal cell; wherein the regulatory regions of the at least one of the two genes is selected from DCC, DAPK, TIMP3, ESR, CCNA1, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, EDNRB and a combination thereof. In particular aspects, the head and neck cancer is head and neck squamous cell carcinoma (HNSCC). Illustrative biological samples include saliva and serum sample.

In one aspect the combination of genes includes at least one gene selected from CCNA1, TIMP3, DCC, DAPK, MGMT, MINT31, p16, PGP9.5, MINT1, CDH1, AIM1, ESR, CCND2 and a combination thereof. In particular, this combination of genes is used when the sample is a saliva sample. The combination of genes includes a panel from about 2 to 25 genes or regulatory regions thereof.

In one aspect the combination of genes includes at least one gene selected from HIC1, PGP9.5, CDH1, CCND2, TIMP3, TGFBR2, AIM1, ESR, CCNA1, DCC, MINT31, p16, RARB and a combination thereof. In particular, this combination of genes is used when the sample is a serum sample. The combination of genes includes a panel from about 2 to 25 genes or regulatory regions thereof.

In one aspect, the hypermethylation is determined using quantitative methylation-specific PCR (Q-MSP). In another aspect, the hypermethylation is detected by detecting decreased expression of the gene. In one aspect, decreased expression is detected by reverse transcription-polymerase chain reaction (RT-PCR).

In another embodiment, the invention provides a method of determining the prognosis of a subject having a head and neck cancer. The method includes determining the methylation state of a gene or the regulatory region of at least two genes in a nucleic acid sample from the subject, wherein the at least two genes or regulatory regions are hypermethylated as compared to the same regions in a corresponding normal cell; wherein the regulatory regions of at least one of the two genes is selected from DCC, DAPK, TIMP3, ESR, CCNA1, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, and EDNRB and a combination thereof; and wherein the hypermethylation of the region as compared to the same region in a corresponding normal cell is indicative of a poor prognosis.

In another embodiment, the invention provides a method for determining whether a subject is responsive to a particular therapeutic regimen including determining the methylation state of a gene or the regulatory region of at least two genes, in a nucleic acid sample from the subject, wherein the at least two genes or regulatory regions are hypermethylated as compared to the same regions in a corresponding normal cell; wherein the regulatory regions of at least one of the two genes is selected from DCC, DAPK, TIMP3, ESR, CCNA1, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, EDNRB and a combination thereof; and wherein the hypermethylation of the region as compared to the same region in a corresponding normal cell is indicative of a subject who may be responsive to the therapeutic regimen.

In one aspect, the therapeutic regimen is administration of a chemotherapeutic agent such as methotrexate, cisplatin/carboplatin, canbusil, dactinomicin, taxol (paclitaxol), a vinca alkaloid, a mitomycin-type antibiotic, a bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine, vindesine, tamoxifen, and mechlorethamine.

In another aspect, the therapeutic regimen is administration of a demethylating agent such as 5-azacytidine or 5-aza-2-deoxycytidine or zebularine. The method of the invention also includes a combination therapeutic approach using a chemotherapeutic agent in combination with a demethylating agent, in any sequence of administration.

In yet another embodiment, the invention provides a kit including an agent that provides a determination of the methylation state of a gene or the regulatory region of at least two genes, and a panel of at least one gene selected from DCC, DAPK, TIMP3, ESR, CCNA1, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, EDNRB and a combination thereof. In one aspect, the combination of genes includes at least CCNA1, TIMP3, DCC, DAPK, MGMT, MINT31, p16, PGP9.5, MINT1, CDH1, AIM1, ESR, CCND2 and a combination thereof.

In another embodiment, the invention provides a kit including an agent that provides a determination of the methylation state of a gene or the regulatory region of at least two genes; and a panel of two or more genes selected from the group consisting of DCC, DAPK, TIMP3, ESR, CCNA1, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, EDNRB and a combination thereof. In one aspect, the combination of genes includes at least HIC1, PGP9.5, CDH1, CCND2, TIMP3, TGFBR2, AIM1, ESR, CCNA1A, DCC, MINT31, p16, RARB and a combination thereof.

### BRIEF DESCRIPTION OF THE DRAWINGS

FIG. 1 is a table which shows the primers and probes designed to specifically amplify the bisulfite-converted DNA for the ACTB gene (SEQ ID NO'S 1-3) and all genes of interest (SEQ ID NO'S 4-66).

FIG. 2 is a table which shows all the possible combinations and results of the genes tested in saliva.

FIG. 3 is a table which shows all the possible combinations and results of the genes tested in serum.

FIG. 4 is a graph which shows the operating characteristic curves for selected panels for saliva samples single point represented the performance of the panel with a positive panel being defined as at least one gene of the panel presented methylation.

FIG. 5 is a graph which shows the operating characteristic curves for selected panels for serum samples single point represented the performance of the panel with a positive panel being defined as at least one gene of the panel presented methylation.

FIGS. 6A-6E are graphs which show patterns of hypermethylation in DNA tumor (case) and DNA salivary rinses (control) for the selected genes. FIG. 6A shows the pattern for the gene MINT31, FIG. 6B shows the pattern for the gene DCC, FIG. 6C shows the pattern for the gene CALCA, FIG. 6D shows the pattern for the gene CCND2, FIG. 6E shows the pattern for the gene RBM6.

FIGS. 7A and 7B are graphs which show a plot of specificity versus sensitivity for head and neck cancer detection on body fluids with FIG. 7A showing the detection in salivary rinses and FIG. 7B showing the detection in serum.

FIGS. 8A-8D are graphs which show the compartment-specific methylation considering methylation patterns on tumor, saliva from controls, and serum from controls for selected genes. The X axis, represents the proportion of methylated cases/tested cases for each sample type. The Y axis represents the quantity of hypermethylation gene of interest/ACTB x 100. FIG. 8A shows methylation of TIMP3. FIG. 8B shows the methylation of DAPK. FIG. 8C shows the methylation of TGFBR2. FIG. 8D shows the methylation of S100A2.

FIG. 9 is a table which demonstrates the analyses based on samples from saliva cases vs. saliva control. The results include the frequency distributions AUC, sensitivity and specificity for each gene and are summarized in the table

FIG. 10 is a table which shows the detection of promoter hypermethylation patterns on saliva pre-treatment (full panel) according to clinical characteristics.

FIG. 11 is a graph which shows the local control rates according to the hypermethylation pattern on saliva pre-treatment (full panel).

FIG. 12 is a graph which shows the overall survival according to the hypermethylation pattern on saliva pre-treatment (full panel).

FIG. 13 is a table which shows the local control and overall survival rates according to the clinical variables tested in Example 2.

FIG. 14 is a table which shows the local control and overall survival rates according to the promoter hypermethylation pattern on saliva pre-treatment in Example 2

FIG. 15 is a table which shows the multivariate analysis for local control and overall survival in Example 2.

FIG. 16 is a graph which shows the quantity of hypermethylation of KIF1A/ACTB×100 found in the saliva of patients with tumor=T (cases), versus the saliva of patients without tumor=N (controls).

FIG. 17 is a graph which shows the quantity of hypermethylation of EDNRB/ACTB×100 found in the saliva of patients with tumor=T (cases), versus the saliva of patients without tumor=N (controls)

FIG. 18 is a graph which shows the quantity of hypermethylation of KIF1A and EDNRB/ACTB×100 found in the saliva of patients with tumor=T (cases), versus the saliva of patients without tumor=N (controls).

### DETAILED DESCRIPTION OF THE INVENTION

The present invention is based on compositions and methods for detecting a cellular proliferative disorder in a subject. The method includes obtaining a nucleic acid-containing sample from the subject; contacting the sample with an agent that provides a determination of the methylation state of at least two genes or associated regulatory region of the gene; identifying aberrant methylation of the regions of the genes or regulatory regions, wherein aberrant methylation is identified as being different when compared to the same regions of the gene or associated regulatory region in a subject not having a cellular proliferative disorder. The method of the invention is also useful for prognostic analyses.

Examples of a cellular proliferative disorder includes non-small cell lung cancer, head and neck carcinoma, lymphoma, melanoma, myeloma, neuroblastoma, glioblastoma, ovarian cancer, pancreatic cancer, prostate cancer, urothelial cancer, breast cancer, colon cancer, thyroid cancer, testicular cancer, tumors of the oral cavity, larynx, pharynx, neck, skull base, salivary glands, and premalignant conditions of the upper aerodigestive tract. Preferably, the cellular proliferative disorder is head and neck squamous cell carcinoma.

In certain embodiments, the gene or regulatory region is two or more genes including those listed here and/or additional genes (the “target genes”). In particular embodiments, at least one gene or regulatory region thereof is selected from DCC, DAPK, TIMP3, ESR, CCNA1, CCND2, MINT31, CDH1, AIM1, MGMT, p16, PGP9.5, RARB, HIC1, RASSF1A, CALCA, TGFBR2, S100A2, RIZ1, RBM6, KIF1 and EDNRB.

As provided herein, hypermethylation may occur in the gene or regulatory region thereof. In some embodiments, the hypermethylation occurs within the regulatory region of the genes identified herein, in particular embodiments, the hypermethylation is in the promoter sequence of the regulatory region. More particularly, the hypermethylation may be in a CpG dinucleotide motif of the promoter.

In another embodiment, there are provided methods for diagnosing a disorder in a subject having or at risk of developing a cell proliferative disorder. The method includes contacting a nucleic acid-containing sample from cells of the subject with an agent that provides a determination of the methylation state of at least one regulatory region of a gene, wherein the at least one regulatory region is hypermethylated in a cell undergoing unregulated cell growth as compared to a corresponding normal cell; and identifying hypermethylation of the regulatory region in the nucleic acid-containing sample, as compared to the same region of the at least one regulatory region in a subject not having the proliferative disorder, wherein hypermethylation is indicative of a subject having or at risk of developing the proliferative disorder.

The term “cell proliferative disorder” as used herein refers to malignant as well as non-malignant cell populations which often differ from the surrounding tissue both morphologically and genotypically. In some embodiments, the cell proliferative disorder is a cancer. In particular embodiments the cancer may be a carcinoma or a sarcoma. A cancer can include, but is not limited to, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, and adenomas. In one aspect, the cancer is head and neck cancer. In another aspect, the head and neck cancer is head and neck squamous cell carcinoma.

The nucleic acid-containing sample for use in the invention methods may be virtually any biological sample that contains nucleic acids from the subject. The biological sample can be a tissue sample which contains 1 to 10,000,000, 1000 to 10,000,000, or 1,000,000 to 10,000,000 somatic cells. However, it is possible to obtain samples that contain smaller numbers of cells, even a single cell in embodiments that utilize an amplification protocol such as PCR. The sample need not contain any intact cells, so long as it contains sufficient material (e.g., protein or genetic material, such as RNA or DNA) to assess methylation status or gene expression levels. In some embodiments the nucleic acid-containing sample is obtained from cells are from a sample selected from the group consisting of a tissue sample, a frozen tissue sample, a biopsy specimen, a surgical specimen, a cytological specimen, whole blood, bone marrow, cerebral spinal fluid, peritoneal fluid, pleural fluid, lymph fluid, serum, mucus, plasma, urine, chyle, stool, ejaculate, sputum, nipple aspirate and saliva. In one aspect the sample is serum and saliva.

A biological or tissue sample can be drawn from any tissue that is susceptible to cancer. For example, the tissue may be obtained by surgery, biopsy, swab, stool, or other collection method. The biological sample for methods of the present invention can be, for example, a sample from colorectal tissue, or in certain embodiments, can be a blood sample, or a fraction of a blood sample such as a peripheral blood lymphocyte (PBL) fraction. Methods for isolating PBLs from whole blood are well known in the art. An example of such a method is provided in the Example section herein. In addition, it is possible to use a blood sample and enrich the small amount of circulating cells from a tissue of interest, e.g., lung, colon, breast, etc. using a method known in the art.

In the present invention, the subject is typically a human, but also can be any mammal, including, but not limited to, a dog, cat, rabbit, cow, rat, horse, pig, or monkey.

Numerous methods for analyzing methylation status of a gene or regulatory region are known in the art and can be used in the methods of the present invention to identify hypermethylation. As illustrated in the Examples herein, analysis of methylation can be performed by bisulfite genomic sequencing.

Bisulfite ions, for example, sodium bisulfite, convert non-methylated cytosine residues to bisulfite modified cytosine residues. The bisulfite ion treated gene sequence can be exposed to alkaline conditions, which convert bisulfite modified cytosine residues to uracil residues. Sodium bisulfite reacts readily with the 5,6-double bond of cytosine (but poorly with methylated cytosine) to form a sulfonated cytosine reaction intermediate that is susceptible to deamination, giving rise to a sulfonated uracil. The sulfonate group can be removed by exposure to alkaline conditions, resulting in the formation of uracil. The DNA can be amplified, for example, by PCR, and sequenced to determine whether CpG sites are methylated in the DNA of the sample. Uracil is recognized as a thymine by Taq polymerase and, upon PCR, the resultant product contains cytosine only at the position where 5-methylcytosine was present in the starting template DNA. One can compare the amount or distribution of uracil residues in the bisulfite ion treated gene sequence of the test cell with a similarly treated corresponding non-methylated gene sequence. A decrease in the amount or distribution of uracil residues in the gene from the test cell indicates methylation of cytosine residues in CpG dinucleotides in the gene of the test cell. The amount or distribution of uracil residues also can be detected by contacting the bisulfite ion treated target gene sequence, following exposure to alkaline conditions, with an oligonucleotide that selectively hybridizes to a nucleotide sequence of the target gene that either contains uracil residues or that lacks uracil residues, but not both, and detecting selective hybridization (or the absence thereof) of the oligonucleotide.

In another embodiment, the gene is contacted with hydrazine, which modifies cytosine residues, but not methylated cytosine residues, then the hydrazine treated gene sequence is contacted with a reagent such as piperidine, which cleaves the nucleic acid molecule at hydrazine modified cytosine residues, thereby generating a product comprising fragments. By separating the fragments according to molecular weight, using, for example, an electrophoretic, chromatographic, or mass spectrographic method, and comparing the separation pattern with that of a similarly treated corresponding non-methylated gene sequence, gaps are apparent at positions in the test gene contained methylated cytosine residues. As such, the presence of gaps is indicative of methylation of a cytosine residue in the CpG dinucleotide in the target gene of the test cell.

Modified products can be detected directly, or after a further reaction which creates products which are easily distinguishable. Means which detect altered size and/or charge can be used to detect modified products, including but not limited to electrophoresis, chromatography, and mass spectrometry. Examples of such chemical reagents for selective modification include hydrazine and bisulfite ions. Hydrazine-modified DNA can be treated with piperidine to cleave it. Bisulfite ion-treated DNA can be treated with alkali. Other means which are reliant on specific sequences can be used, including but not limited to hybridization, amplification, sequencing, and ligase chain reaction. Combinations of such techniques can be used as is desired.

In another example, methylation status may be assessed using real-time methylation specific PCR. For example, the methylation level of the promoter region of one or more of the target genes can be determined by determining the amplification level of the promoter region of the target gene based on amplification-mediated displacement of one or more probes whose binding sites are located within the amplicon. In general, real-time quantitative methylation specific PCR is based on the continuous monitoring of a progressive fluorogenic PCR by an optical system. Such PCR systems are well-known in the art and usually use two amplification primers and an additional amplicon-specific, fluorogenic hybridization probe that specifically binds to a site within the amplicon. The probe can include one or more fluorescence label moieties. For example, the probe can be labeled with two fluorescent dyes: 1) a 6-carboxy-fluorescein (FAM), located at the 5′-end, which serves as reporter, and 2) a 6-carboxy-tetramethyl-rhodamine (TAMRA), located at the 3′-end, which serves as a quencher. When amplification occurs, the 5′-3′ exonuclease activity of the Taq DNA polymerase cleaves the reporter from the probe during the extension phase, thus releasing it from the quencher. The resulting increase in fluorescence emission of the reporter dye is monitored during the PCR process and represents the number of DNA fragments generated.

In other embodiments, hypermethylation can be identified through nucleic acid sequencing after bisulfite treatment to determine whether a uracil or a cytosine is present at specific location within a gene or regulatory region. If uracil is present after bisulfite treatment, then the nucleotide was unmethylated. Hypermethylation is present when there is a measurable increase in methylation.

In an alternative embodiment, the method for analyzing methylation of the target gene can include amplification using a primer pair specific for methylated residues within a the target gene. In these embodiments, selective hybridization or binding of at least one of the primers is dependent on the methylation state of the target DNA sequence. For example, the amplification reaction can be preceded by bisulfite treatment, and the primers can selectively hybridize to target sequences in a manner that is dependent on bisulfite treatment. For example, one primer can selectively bind to a target sequence only when one or more base of the target sequence is altered by bisulfite treatment, thereby being specific for a methylated target sequence.

Other methods are known in the art for determining methylation status of a target gene, including, but not limited to, array-based methylation analysis and Southern blot analysis.

Methods using an amplification reaction can utilize a real-time detection amplification procedure. For example, the method can utilize molecular beacon technology.

In addition, methyl light (Trinh B N, Long T I, Laird P W. 25(4):456-62 (2001), incorporated herein in its entirety by reference), Methyl Heavy (Epigenomics, Berlin, Germany), or SNuPE (single nucleotide primer extension) (See e.g., Watson D., et al., Genet Res. 75(3):269-74 (2000)) can be used in the methods of the present invention related to identifying altered methylation of the genes or regulatory regions provided herein. Additionally, methyl light, methyl heavy, and array-based methylation analysis can be performed, by using bisulfate treated DNA that is then PCR-amplified, against microarrays of oligonucleotide target sequences with the various forms corresponding to unmethylated and methylated DNA.

The degree of methylation in the DNA associated with the gene or genes or regulatory regions thereof, may be measured by fluorescent in situ hybridization (FISH) by means of probes which identify and differentiate between genomic DNAs, which exhibit different degrees of DNA methylation. FISH is described in the Human chromosomes: principles and techniques (Editors, Ram S. Verma, Arvind Babu Verma, Ram S.) 2nd ed., New York: McGraw-Hill, 1995, which is incorporated herein by reference. In this case, the biological sample will typically be any which contains sufficient whole cells or nuclei to perform short term culture. Usually, the sample will be a tissue sample that contains 10 to 10,000, or, for example, 100 to 10,000, whole somatic cells.

In other embodiments, methylation-sensitive restriction endonucleases can be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc III, Ban I, BstN I, Msp I, and Xma I. Examples of the latter are Acc II, Ava I, BssH II, BstU I, Hpa II, and Not I. Alternatively, chemical reagents can be used which selectively modify either the methylated or non-methylated form of CpG dinucleotide motifs.

In some embodiments, hypermethylation of the target gene is detected by detecting decreased expression of the gene. Expression of a gene can be assessed using any means known in the art. Typically expression is assessed and compared in test samples and control samples which may be normal, non-malignant cells. The test samples may contain cancer cells or pre-cancer cells or nucleic acids from them. Methods employing hybridization to nucleic acid probes can be employed for measuring specific mRNAs. Such methods include using nucleic acid probe arrays (microarray technology), in situ hybridization, and using Northern blots. Messenger RNA can also be assessed using amplification techniques, such as RT-PCR. Advances in genomic technologies now permit the simultaneous analysis of thousands of genes, although many are based on the same concept of specific probe-target hybridization. Sequencing-based methods are an alternative; these methods started with the use of expressed sequence tags (ESTs), and now include methods based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest. Moreover, specific proteins can be assessed using any convenient method including immunoassays and immuno-cytochemistry but are not limited to that. Most such methods will employ antibodies which are specific for the particular protein or protein fragments. The sequences of the mRNA (cDNA) and proteins of the target genes of the present invention are known in the art and publicly available.

As used herein, the term “selective hybridization” or “selectively hybridize” refers to hybridization under moderately stringent or highly stringent physiological conditions, which can distinguish related nucleotide sequences from unrelated nucleotide sequences.

As known in the art, in nucleic acid hybridization reactions, the conditions used to achieve a particular level of stringency will vary, depending on the nature of the nucleic acids being hybridized. For example, the length, degree of complementarity, nucleotide sequence composition (for example, relative GC:AT content), and nucleic acid type, i.e., whether the oligonucleotide or the target nucleic acid sequence is DNA or RNA, can be considered in selecting hybridization conditions. An additional consideration is whether one of the nucleic acids is immobilized, for example, on a filter. Methods for selecting appropriate stringency conditions can be determined empirically or estimated using various formulas, and are well known in the art (see, for example, Sambrook et al., supra, 1989).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, for example, high stringency conditions, or each of the conditions can be used, for example, for 10 to 15 minutes each, in the order listed above, repeating any or all of the steps listed.

The term “nucleic acid molecule” is used broadly herein to mean a sequence of deoxyribonucleotides or ribonucleotides that are linked together by a phosphodiester bond. As such, the term “nucleic acid molecule” is meant to include DNA and RNA, which can be single stranded or double stranded, as well as DNA/RNA hybrids. Furthermore, the term “nucleic acid molecule” as used herein includes naturally occurring nucleic acid molecules, which can be isolated from a cell, for example, a particular gene of interest, as well as synthetic molecules, which can be prepared, for example, by methods of chemical synthesis or by enzymatic methods such as by the polymerase chain reaction (PCR), and, in various embodiments, can contain nucleotide analogs or a backbone bond other than a phosphodiester bond.

The terms “polynucleotide” and “oligonucleotide” also are used herein to refer to nucleic acid molecules. Although no specific distinction from each other or from “nucleic acid molecule” is intended by the use of these terms, the term “polynucleotide” is used generally in reference to a nucleic acid molecule that encodes a polypeptide, or a peptide portion thereof, whereas the term “oligonucleotide” is used generally in reference to a nucleotide sequence useful as a probe, a PCR primer, an antisense molecule, or the like. Of course, it will be recognized that an “oligonucleotide” also can encode a peptide. As such, the different terms are used primarily for convenience of discussion.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

General embodiments of the present invention relate to methods and kits used for diagnosing, or evaluating a subject having or at risk of developing head and neck cancer by determining the methylation state of a gene or the regulatory region of at least one gene in a nucleic acid sample from the subject, and wherein at least one gene or regulatory region is hypermethylated as compared to the same region in a corresponding normal cell.

A further embodiment of the present invention includes the use of a plurality of genes or the regulatory regions of the genes in the methods described herein. When more than one gene or regulatory region is used in a method (for example in a panel of gene promoters) the regulatory regions in each of the genes may be identified as hypermethylated as compared to the same region in a corresponding normal cell. One embodiment contemplated a method and or kit used for diagnosing a subject or at risk of developing head and neck cancer by determining the methylation state of at least two genes or the regulatory regions of at least two genes in a nucleic acid sample from the subject, wherein at least two genes or regulatory regions in the two genes are identified as hypermethylated.

The general methods and kits contemplated are based on chemical changes (promoter hypermethylation) that are preferentially detected in the salivary rinse and serum DNA of patients with head and neck cancer and individuals at risk for head and neck cancer.

Examples of references describing methods of detecting a cellular proliferative disorder, such as HNSCC, by determining the methylation state of at least one gene or regulatory region of a gene include U.S. Pat. Nos. 7,214,485; 7,153,657; 7,153,653; 6,893,820; 6,811,982; and 6,617,434.

One embodiment of the present methods includes a set of selected gene promoters that are preferentially chemically altered (methylated) in salivary rinses and serum DNA in association with head and neck cancer risk. Embodiments of the methods include current means of detecting promoter hypermethylation, quantititative methylation specific PCR, as well as other means of determining gene specific promoter hypermethylation. The current gene panel may be modified in an ongoing fashion to include other genes that aid in detection of risk for head and neck cancer.

One embodiment includes methods where the hypermethylation is at a CpG dinucleotide motif in the at least one gene or regulatory region which may be a promoter.

The methods for detecting hypermethylation include but are not limited to: (a) detecting decreased expression of the gene; (b) detecting decreased mRNA of the gene; (c) detecting decreased protein encoded by the gene; and (d) detected by contacting at least a portion of the gene with a methylation-sensitive restriction endonuclease, the endonuclease preferentially cleaving non-methylated recognition sites relative to methylated recognition sites, whereby cleavage of the portion of the gene indicates non-methylation of the portion of the gene provided that the gene comprises a recognition site for the methylation-sensitive restriction endonuclease.

When the method for detecting hypermethylation is performed by detecting decreased mRNA of the gene the decreased expression of the gene may be detected by reverse transcription-polymerase chain reaction (RT-PCR) for example.

In one embodiment a method for detecting hypermethylation is provided by contacting at least a portion of the gene of the cell with a chemical reagent that selectively modifies a non-methylated cytosine residue relative to a methylated cytosine residue, or selectively modifies a methylated cytosine residue relative to a non-methylated cytosine residue; and detecting a product generated by the contacting step. Additionally this method may include the step of hybridization with at least one probe that hybridizes to a sequence comprising a modified non-methylated CpG dinucleotide motif but not to a sequence comprising an unmodified methylated CpG dinucleotide. The method may further include the step of amplification with at least one primer that hybridizes to a sequence comprising a modified non-methylated CpG dinucleotide motif but not to a sequence comprising an unmodified methylated CpG dinucleotide motif thereby forming amplification products. The amplification with at least one primer that hybridizes to a sequence comprising an unmodified methylated CpG dinucleotide motif but not to a sequence comprising a modified non-methylated CpG dinucleotide motif thereby forming amplification products is also contemplated.

An additional embodiment features the detection of hypermethylation by contacting at least a portion of the gene of the cell with a chemical reagent that selectively modifies a non-methylated cytosine residue relative to a methylated cytosine residue, or selectively modifies a methylated cytosine residue relative to a non-methylated cytosine residue; and detecting a product generated by the contacting step. Additionally the product may be detected by a method selected from the group consisting of electrophoresis, hybridization, amplification, primer extension, sequencing, ligase chain reaction, chromatography, mass spectrometry, and combinations thereof.

One embodiment of the present invention is based on the testing and identification of a unique profile of gene promoters that are effective markers for risk for head and neck cancer.

The profiles comprise any of the specified genes alone, or in combination with each other or other non-listed or unknown yet to be discovered gene promoters. The gene promoter panels comprise from 2 to 25 genes or regulatory regions of genes. The panel may comprise, by way of example, 2, 3, 4, 5, 6, 7, 8, 9, 10, 11, 12, 13, 14, 15, 16, 17, 18, 19, 20, 21, 22, 23, 24 or 25 genes or regulatory regions of genes. Preferably the panel will include from 3 to 8 genes or regulatory regions.

A combination of any of the following genes or the regulatory regions of the following genes is included in the present invention: DCC, DAPK, TIMP3, ESR, CCNA1, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, EDNRB.

Another embodiment discloses a panel of promoter hypermethylation markers that have created an improved ability to detect epigenetic changes associated with HNSCC in salivary rinses and serum from patients with HNSCC. Further this panel of promoter hypermethylation markers can be used to anticipate the diagnosis of tumor recurrence by detecting the epigenetic changes associated with HNSCC.

In another embodiment, the invention provides a kit for detecting a cellular proliferative disorder in a subject comprising one or more reagents for detecting the methylation state of at least one gene or regulatory region associated with the following genes: DCC, DAPK, TIMP3, ESR, CCNA1, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, and EDNRB.

Embodiments of the invention include the methods and kits which use both serum and saliva as well as either of them alone.

Another embodiment that uses a saliva sample may selectively include include at least one gene selected from CCNA1, TIMP3, DCC, DAPK, MGMT, MINT31, p16, PGP9.5, MINT1, CDH1, AIM1, ESR, CCND2 and a combination thereof in the methods and kits. Additional embodiments include panels of at least one of these genes either in combination together, or in combination with other non-listed genes.

Another embodiment that uses a serum sample may selectively include include at least one gene selected from HIC1, PGP9.5, CDH1, CCND2, TIMP3, TGFBR2, AIM1, ESR, CCNA1, DCC, MINT31, p16, RARB and a combination thereof in the methods and kits. Additional embodiments include panels of at least one of these genes either in combination together, or in combination with other non-listed genes.

In a further embodiment of the present invention, there are provided methods of identifying a gene deactivated by hypermethylation. The method includes comparing an expression analysis of a cell treated with an agent that reduces methylation to an expression analysis of a control cell not treated with the agent, wherein an increase in expression of a gene is indicative of a gene activated by demethylation. In one aspect, the cell is from a minimally transformed cell line. In some embodiments, the method may further include an expression analysis of a tissue sample and a tumor sample from the same tissue of origin as the treated cell, wherein an increase in expression of a gene in a tumor sample as compared to a normal sample is correlated to the genes activated by demethylation in the treated cell. The method may also include sequence analysis to identify CpG dinucletide motifs in the regulatory region, or particularly the promoter of identified genes. Determination of the methylation status of the identified genes in tumor and corresponding normal tissue samples may also be included.

An additional embodiment of the present invention includes a method of determining the prognosis of a subject having a head and neck cancer by determining the methylation state of a gene or the regulatory region of at least one gene, wherein the gene or the regulatory region is hypermethylated as compared to the same region in a corresponding normal cell; using at least one gene or regulatory region of a gene selected from DCC, DAPK, TIMP3, ESR, CCNA1, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, and EDNRB and a combination thereof. The determination of the prognosis is based on the finding that the hypermethylation of the gene(s) or regulatory region(s) as compared to the same region in a corresponding normal cell(s) is indicative of a poor prognosis.

One embodiment of the present invention includes a method for determining whether a subject is responsive to a particular therapeutic regimen by determining the methylation state of a gene or the regulatory region of at least one gene, in a nucleic acid sample from the subject, wherein the at least one gene or regulatory region is hypermethylated as compared to the same region in a corresponding normal cell; using at least one gene or regulatory region thereof selected from the group consisting of DCC, DAPK, TIMP3, ESR, CCNA1, CCND2. MINT1. MINT31, CDH1, AIM1, MGMT, p16, PGP9.5, RARB, HIC1, RASSF1A, CALCA, TGFBR2, S100A2, RIZ1, RBM6, KIF1, EDNRB and a combination thereof. The determination of whether the subject is responsive to a particular therapeutic regiment is based on determining if hypermethylation of the gene or region above occurs compared to the same region in a corresponding normal cell this may be indicative of a subject who is not responding to the current therapeutic regimen.

In yet another embodiment of the invention, there are provided methods of determining the prognosis of a subject having a cell proliferative disorder. The method includes determining the methylation state of at least one regulatory region of a gene in a nucleic acid sample from the subject, wherein hypermethylation as compared to a corresponding normal cell in the subject or a subject not having the disorder, is indicative of a poor prognosis.

The therapeutic regimen contemplated may include administration of a chemotherapeutic agent selected from methotrexate, cisplatin/carboplatin, canbusil, dactinomicin, taxol (paclitaxol), a vinca alkaloid, a mitomycin-type antibiotic, a bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine, vindesine, tamoxifen, and mechlorethamine.

In one aspect the therapeutic treatment includes administration of a demethylating agent. Such agents are known to those of skill in the art and include 5-azacytidine, 5-aza-2-deoxycytidine or zebularine.

An embodiment of the present invention includes a kit, for practicing any of the methods described above, including an agent that provides a determination of the methylation state of a gene or the regulatory region of at least one gene, and a panel of one or more genes selected from DCC, DAPK, TIMP3, ESR, CCNA1, CCND2. MINT1, MINT31, CDH1, AIM1, MGMT, p16, PGP9.5, RARB, HIC1, RASSF1A, CALCA, TGFBR2, S100A2, RILL, RBM6, KIF1, EDNRB and a combination thereof.

An additional embodiment features a kit, for practicing any of the methods described above, including an agent that provides a determination of the methylation state of a gene or the regulatory region of at least one gene; and a panel of two or more genes selected from DCC, DAPK, TIMP3, ESR, CCNA1, CCND2. MINT1. MINT31, CDH1, AIM1, MGMT, p16, PGP9.5, RARB, HIC1, RASSF1A, CALCA, TGFBR2, S100A2, RIZ1, RBM6, KIF1, EDNRB and a combination thereof.

The following examples are intended to illustrate but not limit the invention.

### Example 1

### Evaluation of Promoter Hypermethylation Detection in Body Fluids as a Screening/Diagnosis Tool for HNSCC

Example 1 discloses identification of an expanded panel of promoter hypermethylation markers which result in an improved ability to detect epigenetic changes associated with HNSCC in salivary rinses and serum from patients with HNSCC. The results of these experiments in Example 1 show differential promoter hypermethylation in HNSCC patients compared with normal individuals in these body fluid compartments.

The Example shows evaluation of the aberrant promoter hypermethylation of candidate tumor suppressor genes as a means to detect epigenetic alterations specific to solid tumors, including HNSCC.

The Example shows promoter regions identified via a candidate gene and discovery approach, and evaluated the ability of an expanded panel of CpG-rich promoters known to be differentially hypermethylated in HNSCC in detection of promoter hypermethylation in serum and salivary rinses associated with HNSCC. A preliminary evaluation via quantitative methylation-specific PCR (Q-MSP) using a panel of 21 genes in a limited cohort of patients with HNSCC and normal controls was performed. Using sensitivity and specificity for individual markers as criteria panels of eight and six genes, respectively, marker panels were selected for use in salivary rinse and serum detection and tested in an expanded cohort including up to 211 patients with HNSCC and 527 normal controls.

Marker panels in salivary rinses showed improved detection when compared with single markers, including a panel with 35% sensitivity and 90% specificity and a panel with 85% sensitivity and 30% specificity. A similar pattern was noted in serum panels, including a panel with 84.5% specificity with 50.0% sensitivity and a panel with sensitivity of 81.0% with specificity of 43.5%. It was also observed that serum and salivary rinse compartments showed a differential pattern of methylation in normal subjects that influenced the utility of individual markers.

It was concluded that Q-MSP detection of HNSCC in serum and salivary rinses using multiple targets may offer improved performance when compared with single markers. Furthermore, compartment-specific methylation in normal subjects affects the utility of Q-MSP detection strategies.

### Materials and Methods

### Tissue Samples.

Samples from 211 HNSCC patients were obtained from patients presenting a previously untreated squamous cell carcinoma from the oral cavity, larynx, or pharynx. Patients were evaluated and enrolled in appropriate protocols in the Department of Otolaryngology-Head and Neck Surgery at Johns Hopkins Medical Institutions (Baltimore, Md.) using appropriate informed consent obtained after institutional review board approval. Tumor, salivary rinse, and serum samples from these patients were collected.

To obtain an accurate determination of methylation status in a cohort of normal individuals, the presence of methylated signal in exfoliated upper aerodigestive cells obtained during a screening study in a control population was assessed. The tissue collected using this technique includes exfoliated epithelial cells from the upper aerodigestive tract, and an exfoliating brush is used to include cells from deep epithelial layers in the oral cavity and oropharynx. This technique allows for a broad sampling of epithelial cells from multiple sites in the upper aerodigestive tract. Salivary rinses were obtained by brushing oral cavity and oropharyngeal surfaces with an exfoliating brush followed by rinse and gargle with 20 mL normal saline solution. Cellular material from the brushing was released into the saline rinse and centrifuged to obtain a cell pellet after supernatant was discarded.

The control population consisted of subjects enrolled in a community screening study for head and neck cancer approved by the Johns Hopkins institutional review board. The experimental protocol was approved by the Johns Hopkins Medical Institutions Institutional Review Board and informed consent was obtained from all enrolled subjects. For the control population, a salivary rinse and blood sample were collected. All subjects were administered a confidential written survey of risk factors for upper aerodigestive tract malignancies, including alcohol and tobacco use as well as the presence of co-morbid illnesses. Smoking was defined as use of tobacco, chewable or smoked, for at least 1 year continuously. Heavy alcohol use was defined as intake of more than two alcoholic drinks per day. A thorough head and neck examination, including cranial nerve function; palpation of cervical, thyroid, and parotid nodal basins; visual inspection and palpation of the oral cavity and oropharynx; and indirect mirror laryngoscopy or flexible fiberoptic laryngoscopy, was done.

For the panel analysis, we excluded those individuals presenting with premalignant or malignant lesions at head and neck area (n=33), past history of cancer regardless of site (n=57), those who were diagnosed of any cancer regardless of site during follow-up (n=62), and those not reachable by phone follow-up (n=119). A total of 527 individuals were included as control population in this study. For purposes of the invention, it should be understood that the panels and methods described herein are useful in individuals presenting with premalignant or malignant lesions at head and neck area, past history of cancer regardless of site, or those who were diagnosed of any cancer regardless of site during follow-up.

### DNA Extraction.

DNA obtained from tumor, salivary rinses, and serum samples was extracted by digestion with 50 μg/mL proteinase K (Boehringer) in the presence of 1% SDS at 48° C. overnight followed by phenol/chloroform extraction and ethanol precipitation.

### Bisulfite Treatment.

DNA from tissue samples was subjected to bisulfite treatment as described previously (Herman et al., Proc Natl Acad Sci USA 1996; 93:9821-6). Briefly, 2 μg of genomic DNA were denatured in 0.2 mol/L NaOH for 20 min at 50° C. The denatured DNA was diluted in 500 μL of freshly prepared solution of 10 mmol/L hydroquinone and 3 mol/L sodium bisulfite and incubated for 3 h at 70° C. After incubation, the DNA sample was desalted through a column (Wizard DNA Clean-Up System, Promega), treated with 0.3 mol/L NaOH for 10 min at room temperature, and precipitated overnight with ethanol. The bisulfite-modified genomic DNA was resuspended in 120 μL H₂O and stored at −80° C.

### Quantitative Methylation-Specific PCR.

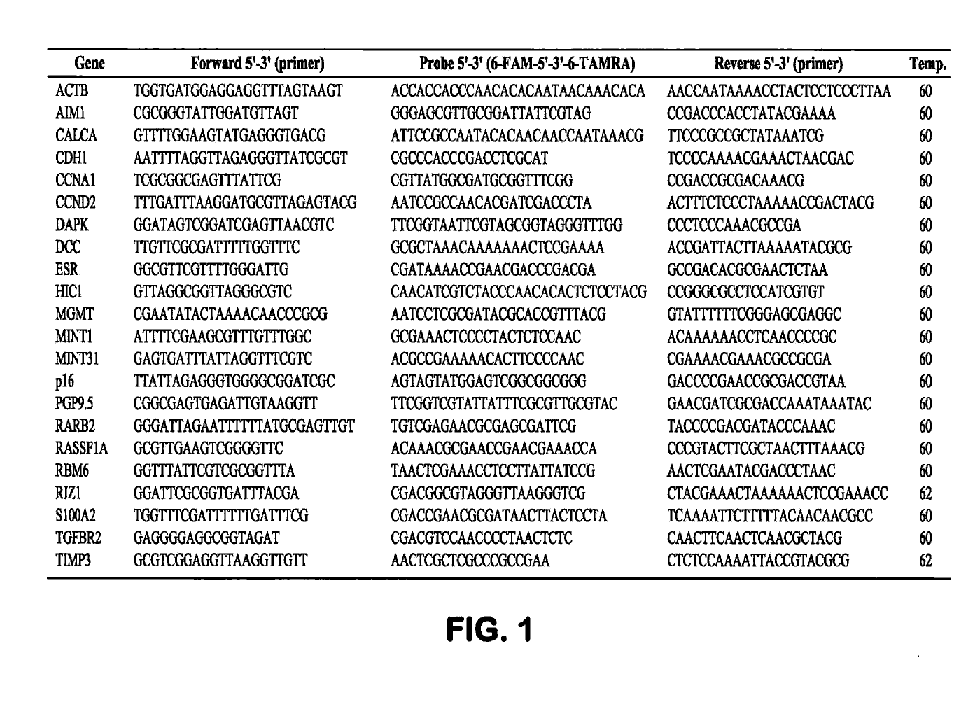

The bisulfite-modified DNA was used as a template for fluorescence-based real-time PCR as described previously (Harden et al., Clin Cancer Res 2003; 9:1370-5). In brief, primers and probes were designed to specifically amplify the bisulfite-converted DNA for the ACTB gene and all genes of interest (primers and probes sequences are available displayed in FIG. 1). The ratios between the values of the gene of interest and the internal reference gene (ACTB), which was obtained by Taqman analysis and take into account the PCR efficiency, were used as a measure for representing the relative quantity of methylation in a particular sample (value for the gene of interest/value for the reference gene x 100). Fluorogenic PCRs were carried out in a reaction volume of 20 μL consisting of 600 nmol/L of each primer; 200 μmol/L of probe; 0.75 unit of platinum Taq polymerase (Invitrogen); 200 μmol/L of each dATP, dCTP, dGTP, and dTTP; 200 nmol/L of ROX Reference Dye (Invitrogen); 16.6 mmol/L ammonium sulfate; 67 mmol/L Trizma (Sigma); 6.7 mmol/L magnesium chloride; 10 mmol/L mercaptoethanol; and 0.1% DMSO. Three microliters of treated DNA solution were used in each real-time MSP reaction. Amplifications were carried out in 384-well plates in a 7900 Sequence Detector System (Perkin-Elmer Applied Biosystems). Thermal cycling was initiated with a first denaturation step at 95° C. for 2 min followed by 45 cycles of 95° C. for 15 s and 60° C. or 62° C. for 1 min. Leukocytes from a healthy individual were methylated in vitro with excess SssI methyltransferase (New England Biolabs) to generate completely methylated DNA, and serial dilutions of this DNA were used for constructing the calibration curves on each plate. Each reaction was done in triplicate; the average of the triplicate was considered for analysis. Results for Q-MSP was analyzed considering the quantity of methylation (normalized by ACTB) as well as considering methylation as a binary event, in which any quantity of methylation in a sample would be considered as positive for methylation.

### Target Gene Selection.

Genes selected for this study came from three different sources: (a) genes with promoters that are reported as hypermethylated in HNSCC [DCC, p16(INK4A), CDH1, MGMT, DAPK, RASSF1A, RARB, and RIZ1]; also including two genome regions known to have a differentially methylated pattern in some tumors (MINT1 and MINT31;); (b) genes with promoters that are reported as hypermethylated in other solid tumors (CCND2, CALCA, TGFBR2, HIC1, S100A2, TIMP3, and ESR); and (c) gene discovery using expression microarray-based approach via unmasking of expression (CCNA1, PGP9.5, AIM1, and RBM6).

### Steps for Gene Evaluation in the Study.

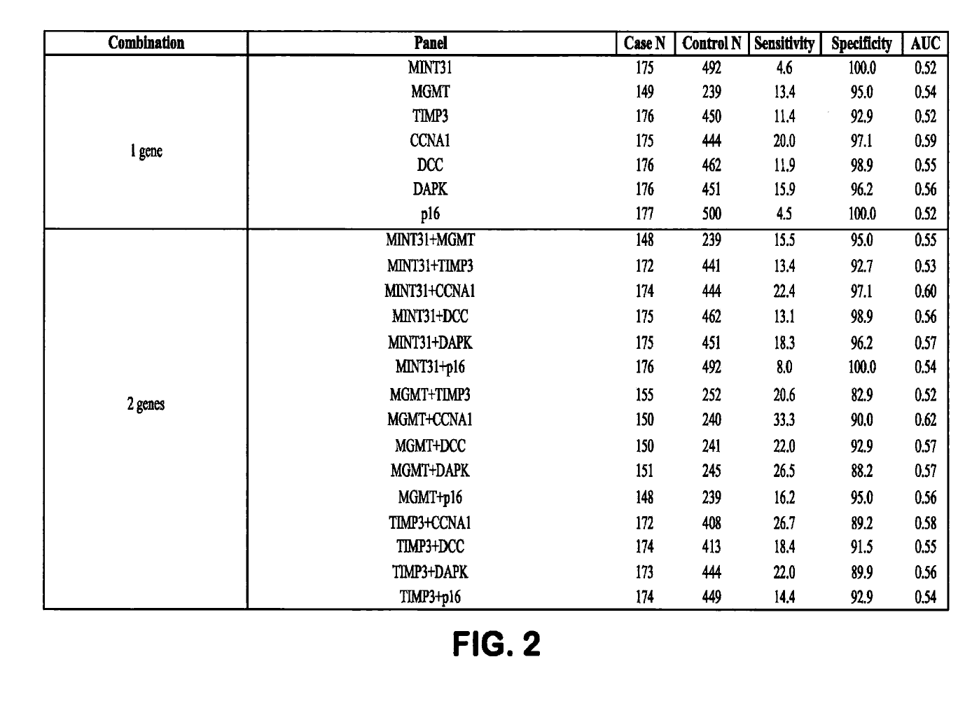

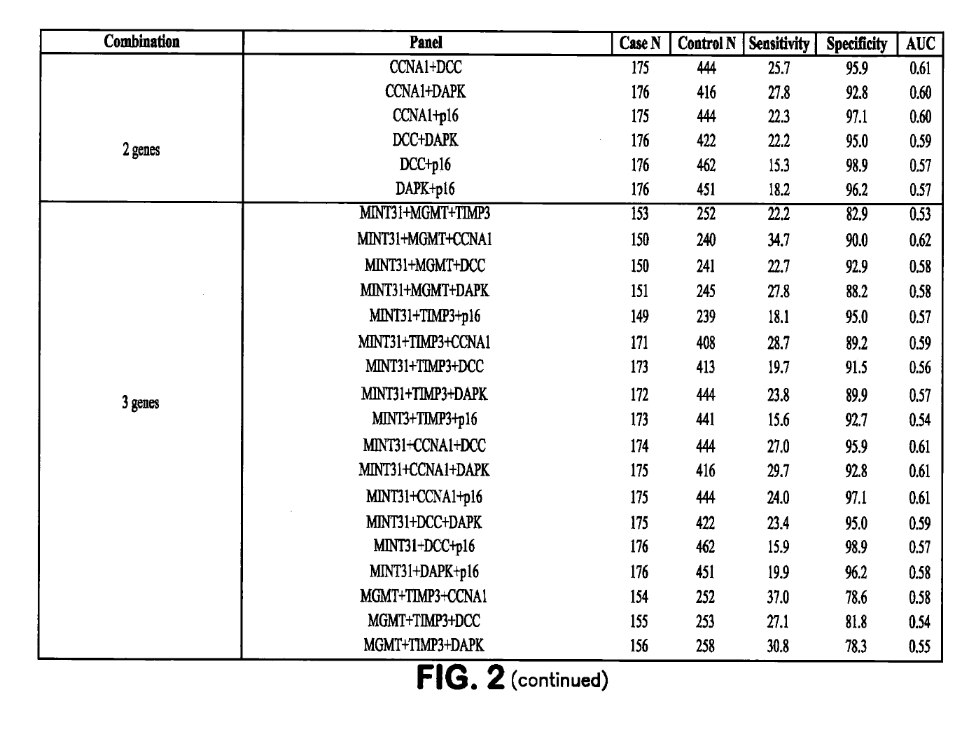

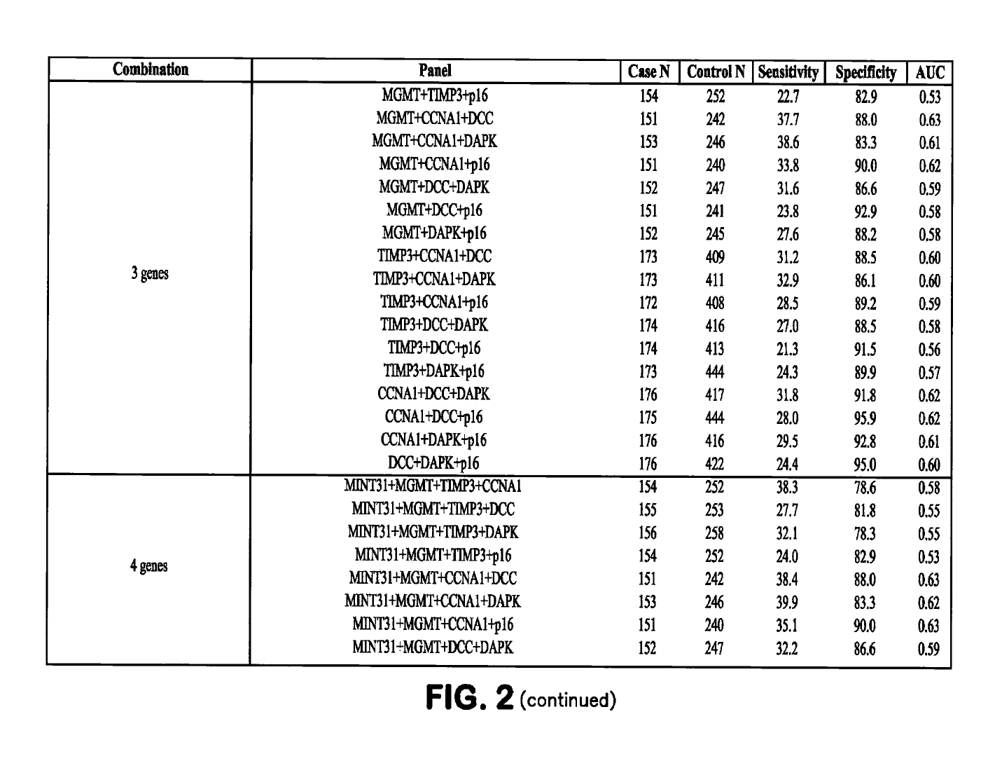

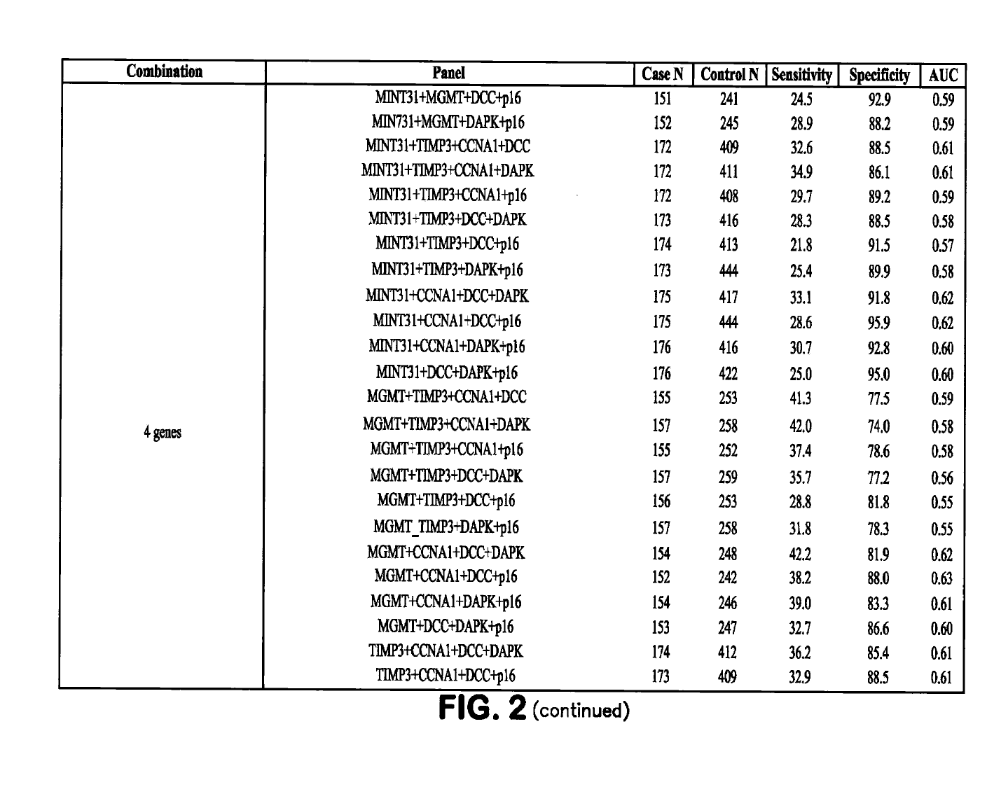

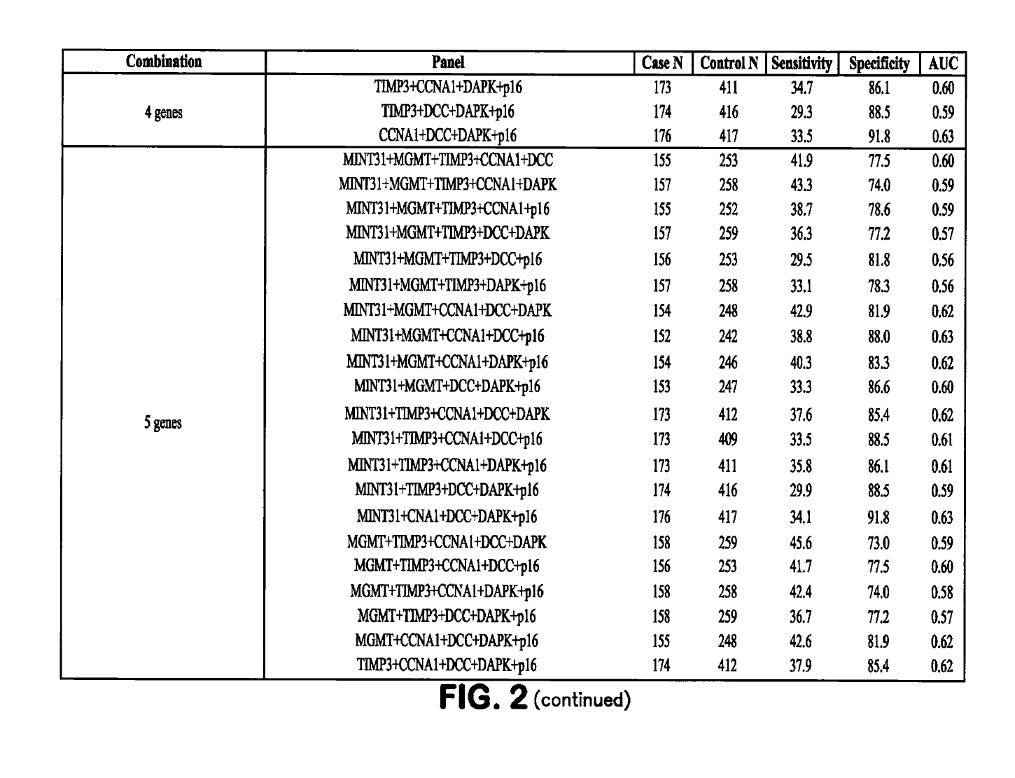

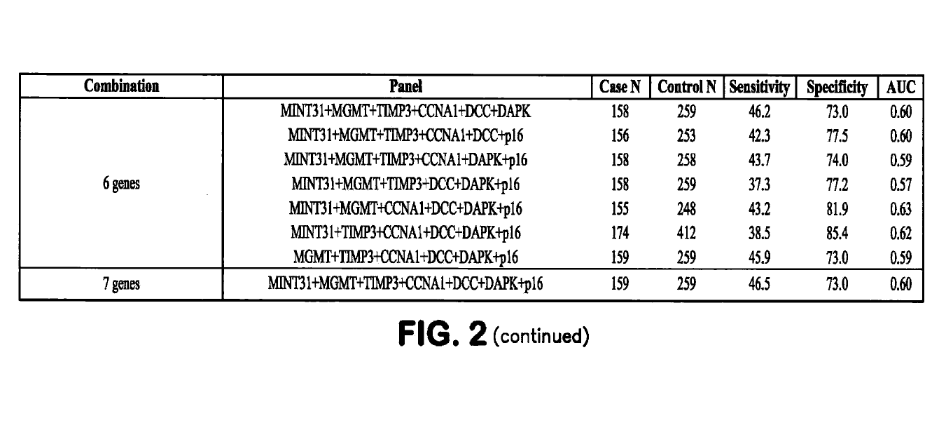

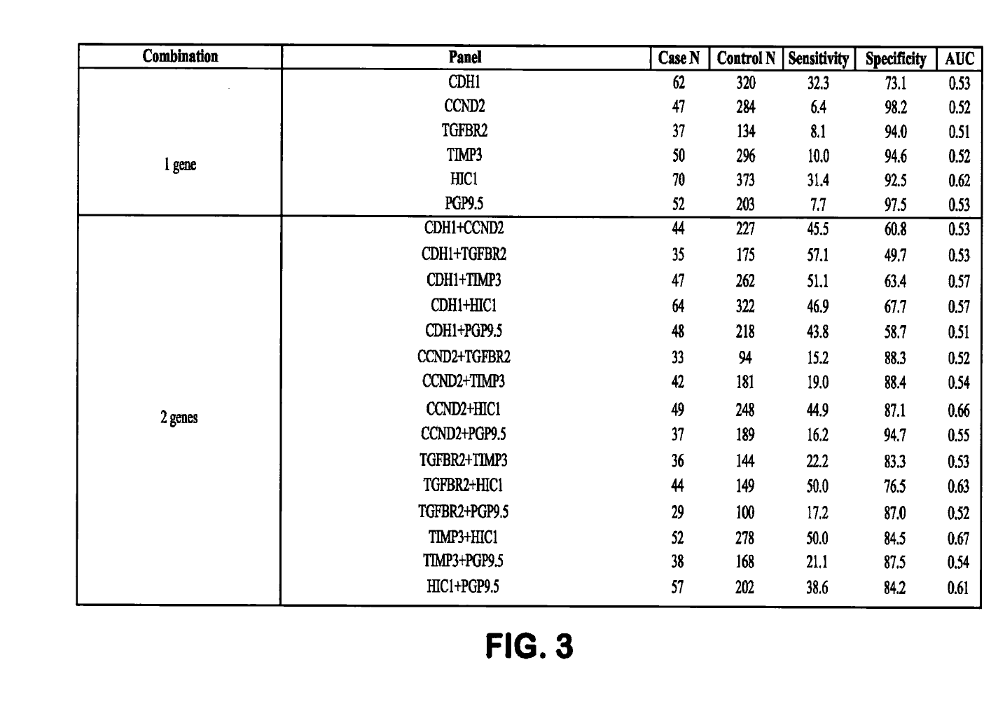

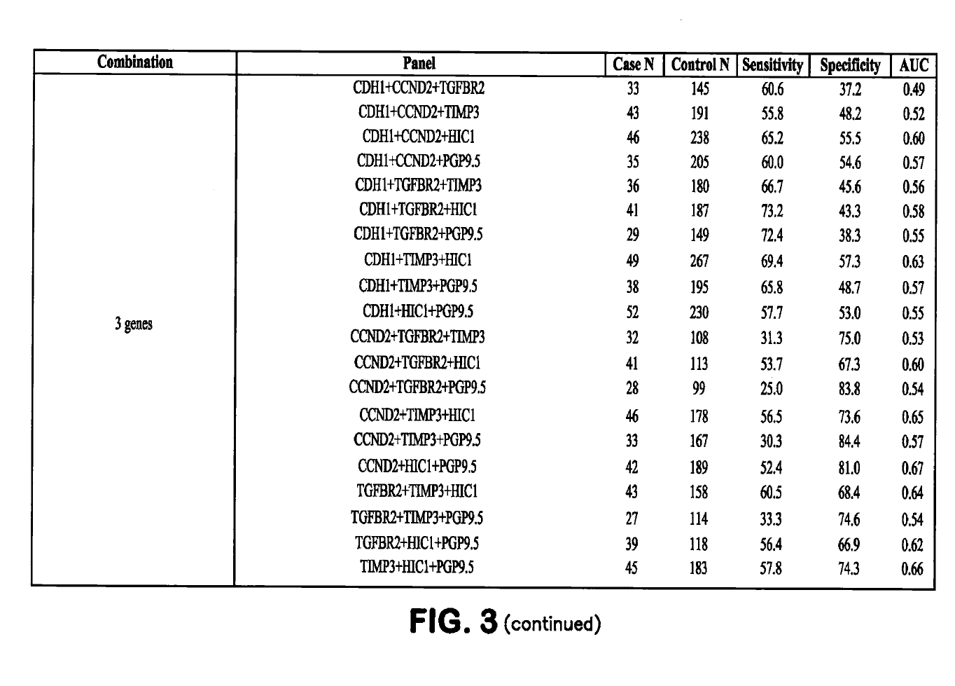

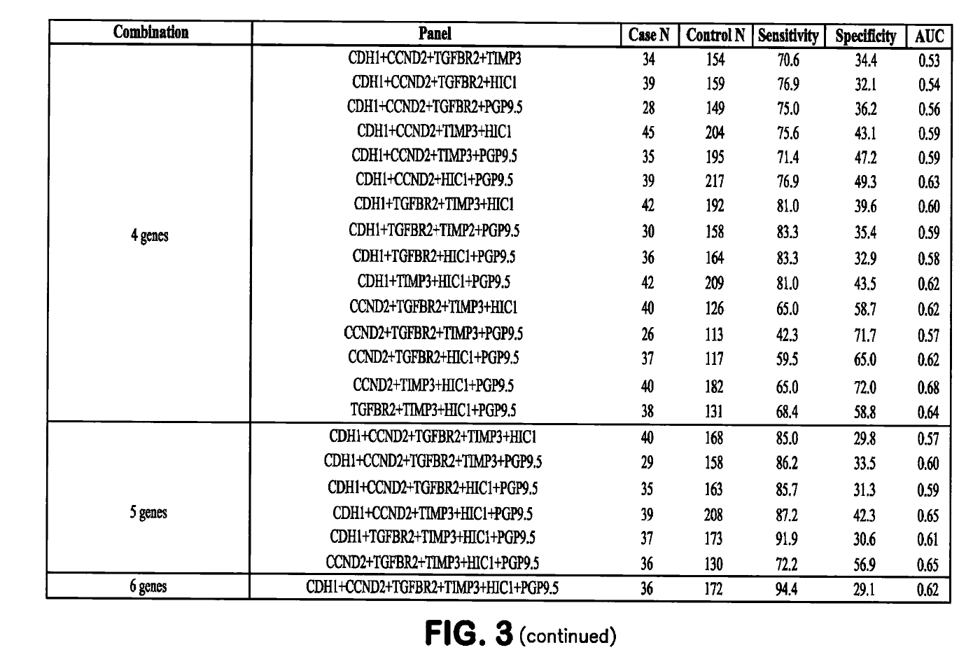

Due to the scarcity of DNA quantity after bisulfite treatment of many samples and the number of genes selected, it would be virtually impossible to evaluate all possible candidate genes in all samples (the exact number of cases considered for each analysis is described in FIGS. 2 and 3). Therefore, a step by step selection with interim statistical analysis, and then a more limited set of “best” genes was used in an expanded cohort of samples. The first step involved a screening evaluation, designed to eliminate targets that had an inappropriately high frequency of promoter hypermethylation in normal, control samples. Elimination of these targets with high rates of promoter hypermethylation in normal control samples facilitated efficient use of limited sample material and allowed for an early definition of higher value markers. A screening evaluation was performed by evaluating candidate genes by comparing tumor samples (cases) with salivary rinses or serum (from controls) in a limited, random subset of both the patient and controls. A screening evaluation directed at the serum and salivary rinse compartments was performed by comparison between salivary rinses (case) from salivary rinses (control) and serum (case) from serum (control) in additional limited sets of HNSCC patients and controls. For this first step of interim analysis, a subset of samples (those with higher concentration of DNA) from the complete cohort was used. Significance was based on area under the curve (AUC) from receiving operating characteristic analysis, sensitivity, and specificity of that particular gene in differentiating the tumor samples (cases) from salivary rinses or serum (controls) for determination of a salivary rinse marker panel, with similar criteria when differentiating salivary rinses (case) from salivary rinses (control) or serum (case) from serum (control).

Markers that fit selection criteria in initial analysis were then used in an expanded analysis of all available tissue from HNSCC patient cases and control subjects. Complete coverage of every sample for every possible methylation marker was not possible due to either limited sample collection or a low quantity of total extracted DNA (particularly for a small proportion of serum samples).

### Statistical Analysis.

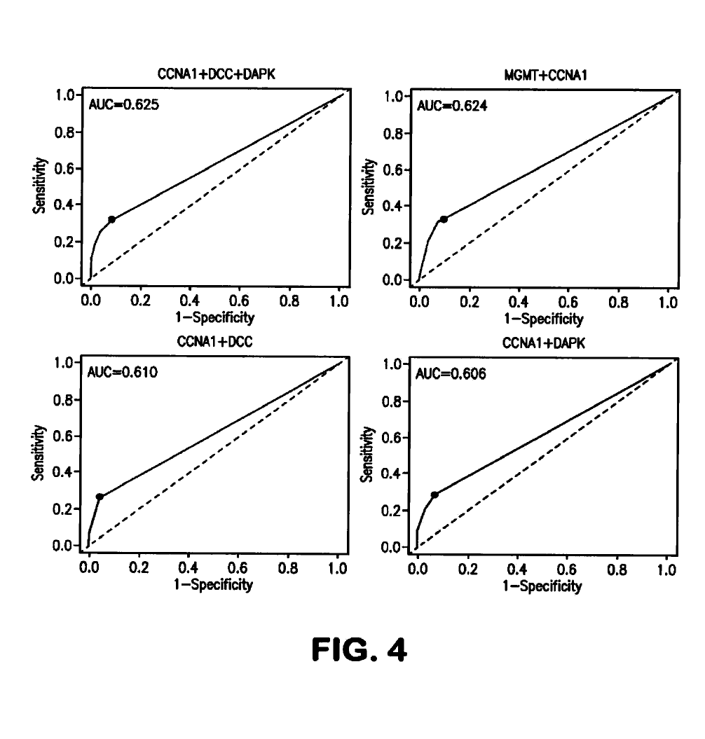

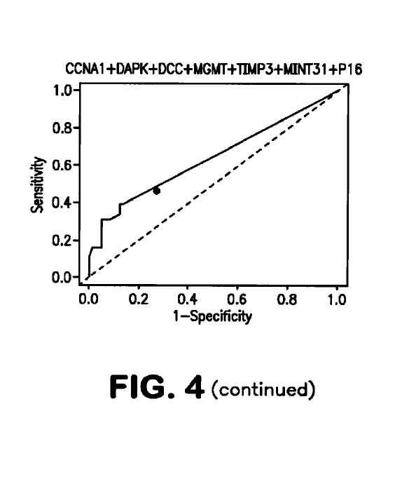

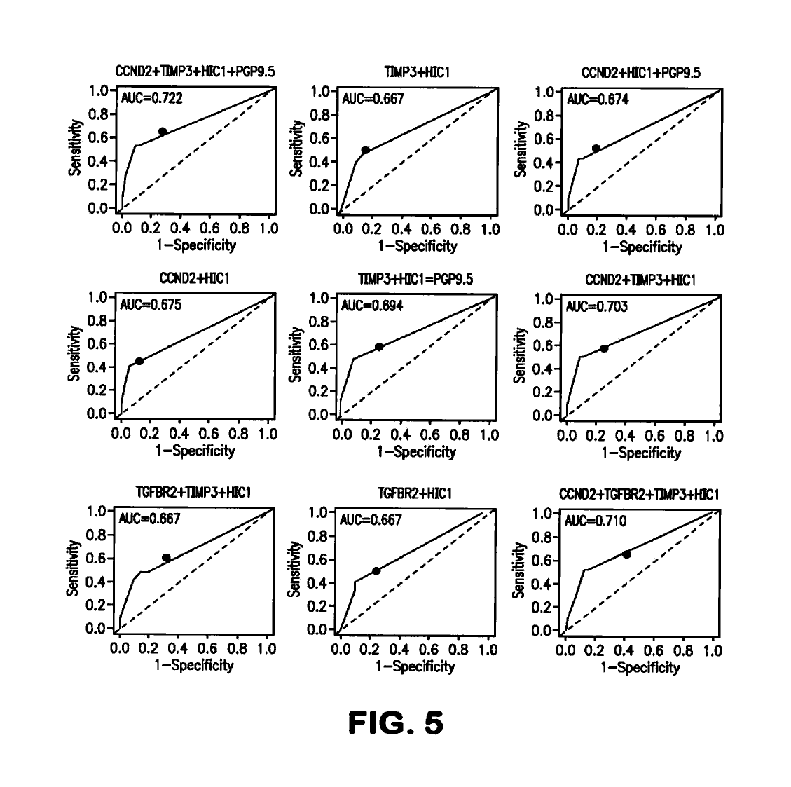

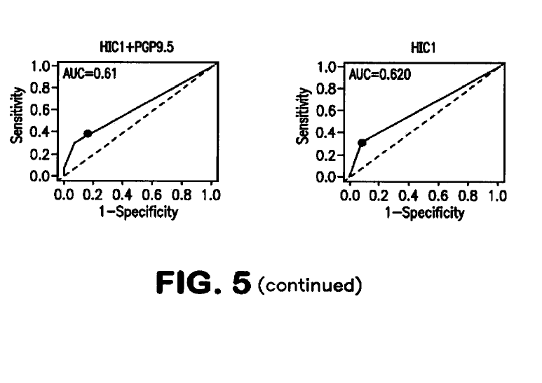

A total of 21 informative genes were considered for this study. Hypermethylation of each gene was treated as a binary variable (methylation versus no methylation) by dichotomizing each gene at zero. Proportions of gene methylation were compared between tumor samples (from cases) and salivary rinses or serum samples (from controls) using Fisher's exact test. Sensitivity and specificity of each individual gene in detecting HNSCC were calculated along with 95% confidence intervals (95% CI), where sensitivity was defined as the number of true-positive results divided by the number of true-positive plus false-negative results and specificity was defined as the number of true-negative results divided by the number of true-negative plus false-positive results. We evaluated all possible combinations of the selected markers for both saliva and serum samples in the expanded cohort, where a positive panel was defined as at least one gene of the panel being methylated (See FIGS. 2 and 3). Sensitivity and specificity were calculated along with 95% CIs. The AUC, an index of predictive power, was also provided. The potential of the confounding effect of the covariates, including age, gender, and tobacco and alcohol assumption, was assessed using stratified analysis. In the meantime, the performances of the selected panels were explored using multivariable logistic regression method. A receiving operating characteristic curve was constructed by plotting the sensitivity (true-positive rate) against 1-specificity (false-positive rate). Internal validation of the logistic regression models was done by using an approximation to the leave-one-out jackknife procedure implemented with Statistical Analysis System software package. Receiving operating characteristic curves for some selected panels based on the method of multivariable logistic regression modeling were constructed for both salivary rinses and serum samples, where the single point represented the performance of the panel with a positive panel being defined as at least one gene of the panel presented methylation (FIGS. 4 and 5). All statistical tests were two sided with P≦0.05 considered statistically significant. All analyses were done using Statistical Analysis System software (version 9.1; SAS Institute, Inc.) and R package (version 1.9.1).

### Results

### HNSCC Patients and Control Population Characteristics.

HNSCC patients were mainly males (75.5%) and Caucasians (78.1%), with ages ranging from 32 to 89 years (median, 57.8 years). Alcohol or tobacco consumption (current or past) was found in 71.3% and 81.7%, respectively.

The control population (subjects enrolled in a community screening study) was mainly males (59.6%) and Caucasians (78.6%), with ages ranging from 18 to 94 years (median, 61.0 years). Alcohol or tobacco consumption (current or past) was found in 79.8% and 61.8%, respectively.

### Initial Screening: Tumor (Case) Versus Salivary Rinses (Control).

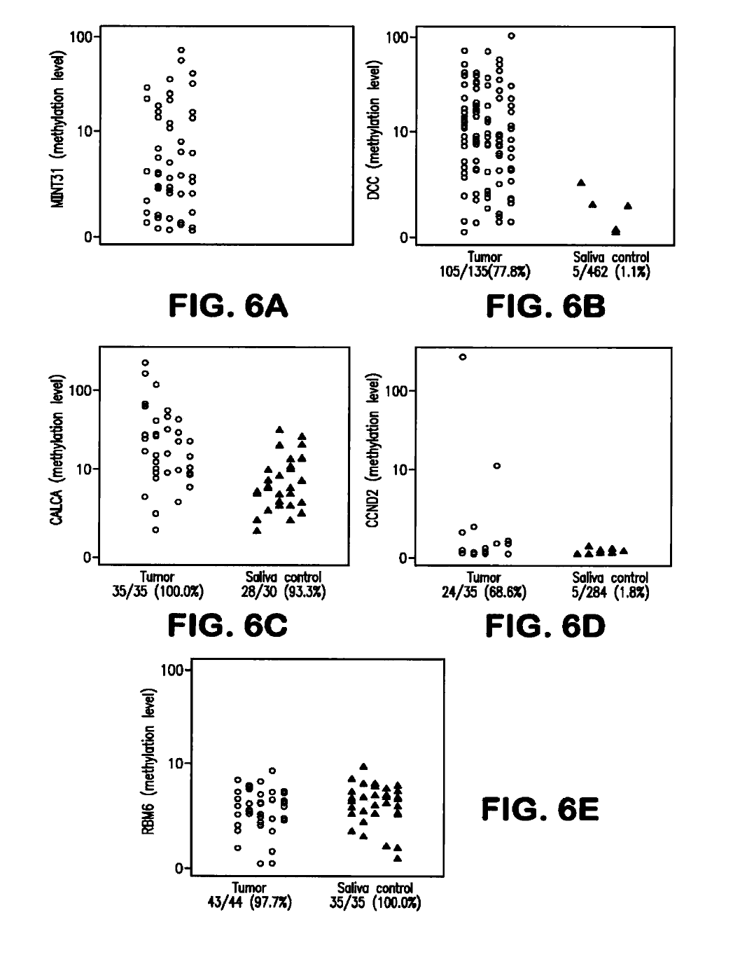

As a first step, the specificity and sensitivity comparing the presence of promoter hypermethylation of selected genes in tumor DNA (from cases) and salivary rinse DNA (from controls) was evaluated. Although these are not identical tissues, this method was used because (a) discovered targets were to be subjected to additional validation using a second comparison of salivary rinses from both patient cohorts, (b) the collection method performed used an exfoliating brush that removes deeper layers of oral and oropharyngeal mucosa, and (c) formal biopsy of the >400 non-cancer patients was not logistically feasible. Distinct methylation patterns were noted as follows: (a) methylation was detected only in HNSCC but not in controls: p16, MINT31, and RASSF1A; (b) a higher frequency and quantity of methylation was noted in HNSCC compared with controls with absent methylation in a subset of control samples: DCC, DAPK, CCNA1, TIMP3, MGMT, AIM1, ESR, MINT1, CDH1, RARB, PGP9.5, and HIC1; (c) a higher frequency of methylation was noted in HNSCC compared with controls but in a similar quantity in both groups: CCND2; (d) a similar frequency of methylation was noted in both groups (tumor and salivary rinses); however, a quantitative difference between groups was noted: CALCA; and (e) methylation was noted in both HNSCC and controls at a similar frequency with no difference in methylation levels: RIZ1, TGFBR2, S100A2, and RBM6. Examples of these patterns are shown on FIG. 6, with overall results shown in Table 1. Twelve genes whose methylation was highly associated with HNSCC, with local recurrences ranging from 1.1 to 70.7 were defined.

TABLE 1
Comparison of hypermethylation detection on tumor DNA
(HNSCC patients) and salivary rinse samples (controls)

| | | Salivary rinses, | | Sensitivity, | Specificity, |
|---|---|---|---|---|---|
| Gene | Tumor, case (n) | control (n) | P* | % (95% CI) | % (95% CI) |
| DCC | 135 | 462 | <0.0001 | 77.8 (69.8-84.5) | 98.9 (97.5-99.7) |
| DAPK | 136 | 451 | <0.0001 | 75.0 (66.9-82.0) | 96.2 (94.0-97.8) |
| TIMP3 | 138 | 450 | <0.0001 | 73.9 (65.8-81.0) | 92.9 (90.1-95.1) |
| ESR | 35 | 119 | <0.0001 | 60.0 (42.1-76.1) | 98.3 (94.1-99.8) |
| CCNA1 | 102 | 444 | <0.0001 | 61.8 (51.6-71.2) | 97.1 (95.1-98.4) |
| CCND2 | 35 | 97 | <0.0001 | 68.6 (50.7-83.2) | 89.7 (81.9-94.7) |
| MINT1 | 87 | 296 | <0.0001 | 90.8 (82.7-96.0) | 66.2 (60.5.71.6) |
| MINT31 | 136 | 492 | <0.0001 | 36.8 (28.7-45.5) | 100 (99.4-100) |
| CDH1 | 77 | 116 | <0.0001 | 90.9 (82.2-96.3) | 37.9 (29.1-47.4) |
| AIM1 | 77 | 73 | <0.0001 | 23.4 (14.5-34.4) | 98.6 (92.6-100) |
| MGMT | 44 | 239 | <0.0001 | 22.7 (11.5-37.8) | 95.0 (91.4-97.4) |
| p16 | 136 | 500 | <0.0001 | 13.2 (8.0-20.1) | 100 (99.4-100) |
| PGP9.5 | 45 | 112 | 0.004 | 91.1 (78.8-97.5) | 30.4 (22.0-39.8) |
| RARB | 44 | 35 | 0.005 | 79.6 (64.7-90.2) | 51.4 (34.0-68.6) |
| HIC1 | 45 | 46 | 0.026 | 100 (93.6-100) | 13.0 (4.9-26.3) |
| RASSF1A | 44 | 35 | 0.063 | 11.4 (3.8-24.6) | 100 (91.8-100) |
| CALCA | 35 | 30 | 0.209 | 100 (91.8-100) | 6.7 (0.8-22.1) |
| TGFBR2 | 11 | 44 | 0.266 | 54.6 (23.4-83.3) | 25.0 (13.2-40.3) |
| S100A2 | 44 | 35 | 0.398 | 95.5 (84.5-99.4) | 11.4 (3.2-26.7) |
| RIZ1 | 44 | 35 | 0.694 | 6.8 (1.4-18.7) | 88.6 (73.3-96.8) |
| RBM6 | 44 | 35 | >0.999 | 97.7 (88.0-99.9) | 0 (0-8.2) |

*Fisher's exact test for the association between gene methylation and HNSCC.

### Initial Screening: Salivary Rinse (Case) Versus Salivary Rinse (Control).

Based on the above results, genes that could distinguish tumor samples (case) from salivary rinse samples (control) for binary results (either presence or absence of methylation) and an AUC>0.60 and at least 90% specificity or sensitivity were selected for further testing on salivary rinses in a limited cohort of HNSCC patients. These markers included CCNA1, DAPK, DCC, MGMT, TIMP3, MINT31, p16, PGP9.5, AIM1, ESR, CCND2, MINT1, and CDH1. It was observed that, in general, although some genes were quite specific for HNSCC compared with DNA from salivary rinses (controls), the sensitivity of these makers for detection of HNSCC in the salivary rinses of HNSCC patients was quite low (Table 2A). It was noted that seven genes now showed local recurrences associated with HNSCC, with ranges from 1.2 to 10.8.

### HNSCC Detection in Salivary Rinses in Expanded Cohort.

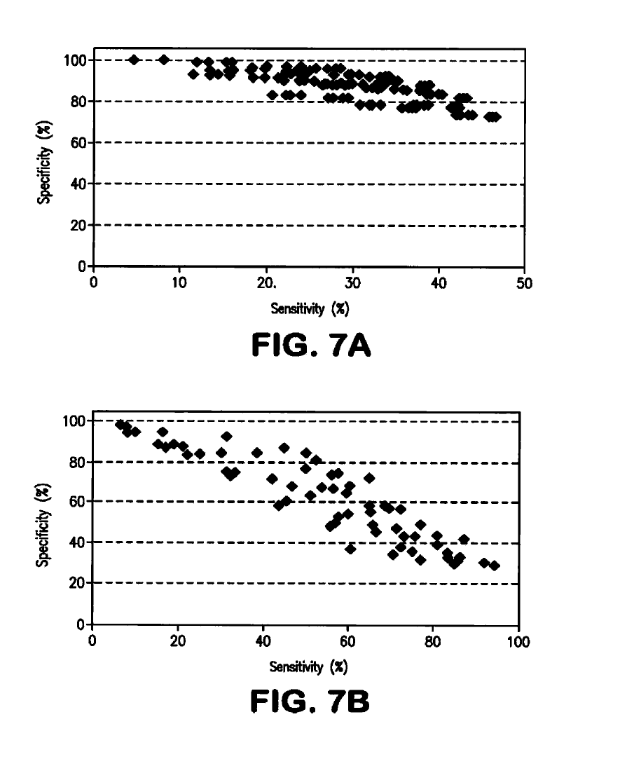

Based on the above results, the genes that could better distinguish the HNSCC patient salivary rinse from control salivary rinse, with an AUC>0.50 and at least 90% specificity and 10% sensitivity, were selected to be tested in combination for the expanded cohort. Also tested were MINT31 and p16, as both markers were 100% specific. The results for AUC, sensitivity, and specificity for all possible combinations with the selected genes are included in the Supplementary Table shown as FIG. 2), and the plot for sensitivity and specificity for those combinations is shown on FIG. 7A. The best performing combination panels are presented in Table 2B, and it was observed that some panels showed up to 91.8% specificity with 34.1% sensitivity. Specificity was observed as high as 97.1% in some combination of genes; however, for these combinations, sensitivity ranged from 22.3% to 24.0%. (See also Carvalho et al., Clin. Cancer Res. 2008:14(1), 97-107)

TABLE 2
Comparison of hypermethylation detection on salivary rinse samples
(HNSCC patients) and salivary rinse samples (controls)
A. Individual gene evaluation

| | | | Sensitivity, | Specificity, |
|---|---|---|---|---|
| Gene | Case (n) | Control (n) | % (95% CI) | % (95% CI) |
| CCNA1 | 175 | 444 | 20.0 (14.3-26.7) | 97.1 (95.1-98.4) |
| DAPK | 176 | 451 | 15.9 (10.8-22.2) | 96.2 (94.0-97.8) |
| DCC | 176 | 462 | 11.9 (7.5-17.7) | 98.9 (97.5-99.7) |
| MGMT | 149 | 239 | 13.4 (8.4-20.0) | 95.0 (91.4-97.4) |
| TIMP3 | 176 | 450 | 11.4 (7.1-17.0) | 92.9 (90.1-95.1) |
| MINT31 | 175 | 492 | 4.6 (2.0-8.8) | 100.0 (99.4-100) |
| p16 | 177 | 500 | 4.5 (2.0-8.7) | 100.0 (99.4-100) |
| PGP9.5 | 34 | 112 | 82.4 (65.5-93.2) | 30.4 (22.0-39.8) |
| AIM1 | 23 | 73 | 4.4 (0.1-22.0) | 98.6 (92.6-100) |
| ESR | 33 | 119 | 3.0 (0.08-15.8) | 98.3 (94.1-99.8) |
| CCND2 | 136 | 97 | 7.4 (3.6-13.1) | 89.7 (81.9-94.9) |
| MINT1 | 131 | 296 | 35.1 (27.0-43.9) | 66.2 (60.5-71.6) |
| CDH1 | 66 | 116 | 30.3 (19.6-42.9) | 37.9 (29.1-47.4) |

B. Best combination of genes for hypermethylation HNSCC detection on salivary rinses

| | | | Sensitivity, | Specificity, |
|---|---|---|---|---|
| Panel | Case (n) | Control (n) | % (95% CI) | % (95% CI) |
| CCNA1 + DCC + DAPK + p16 | 176 | 417 | 33.5 (26.6-41.0) | 91.8 (88.8-94.3) |
| MINT31 + CCNA1 + DCC + DAPK + p16 | 176 | 417 | 34.1 (27.1-41.6) | 91.8 (88.8-94.3) |
| MINT31 + MGMT + CCNA1 + p16 | 151 | 240 | 35.1 (27.5-43.3) | 90.0 (85.5-93.5) |
| MlNT31 + CCNA1 + DCC + p16 | 175 | 444 | 28.6 (22.0-35.9) | 95.9 (93.7-97.6) |
| CCNA1 + DCC + p16 | 175 | 444 | 28.0 (21.5-35.3) | 95.9 (93.7-97.6) |
| MINT31 + CCNA1 + DAPK + p16 | 176 | 416 | 30.7 (24.0-38.1) | 92.8 (89.9-95.1) |
| CCNA1 + DCC + DAPK | 176 | 417 | 31.8 (25.0-39.3) | 91.8 (88.8-94.3) |
| MINT31 + CCNA1 + DCC + DAPK | 175 | 417 | 33.1 (26.2-40.6) | 91.8 (88.8-94.3) |
| MINT31 + MGMT + CCNA1 | 150 | 240 | 34.7 (27.1-42.9) | 90.0 (85.5-93.5) |
| MGMT + CCNA1 + p16 | 151 | 240 | 33.8 (26.3-41.9) | 90.0 (85.5-93.5) |
| MGMT + CCNA1 | 150 | 240 | 33.3 (25.9-41.5) | 90.0 (85.5-93.5) |
| CCNA1 + DCC | 175 | 444 | 25.7 (19.4-32.9) | 95.9 (93.7-97.6) |
| MINT31 + CCNA1 + DCC | 174 | 444 | 27.0 (20.6-34.3) | 95.9 (93.7-97.6) |
| MINT31 + CCNA1 + DAPK | 175 | 416 | 29.7 (23.1-37.1) | 92.8 (89.9-95.1) |
| CCNA1 + DAPK + p16 | 176 | 416 | 29.5 (22.9-36.9) | 92.8 (89.9-95.1) |
| MINT31 + CCNA1 + p16 | 175 | 444 | 24.0 (17.9-31.0) | 97.1 (95.1-98.4) |
| CCNA1 + DAPK | 176 | 416 | 27.8 (21.4-35.1) | 92.8 (89.9-95.1) |
| MINT31 + CCNA1 | 174 | 444 | 22.4 (16.5-29.3) | 97.1 (95.1-98.4) |
| CCNA1 + p16 | 175 | 444 | 22.3 (16.4-29.2) | 97.1 (95.1-98.4) |
| DCC + DAPK + p16 | 176 | 422 | 24.4 (18.3-31.5) | 95.0 (92.5-96.9) |
| MINT31 + DCC + DAPK + p16 | 176 | 422 | 25.0 (18.8-32.1) | 95.0 (92.5-96.9) |

### Initial Screening: Tumor (Case) Versus Serum (Control).

Again, as a first step in developing a detection panel for serum, the specificity and sensitivity by comparing promoter hypermethylation of selected genes in tumor DNA versus serum DNA from control subjects without cancer was evaluated. Distinct methylation patterns were noted as follows: (a) hypermethylation was detected only in HNSCC but not in controls: p16, MINT31, CCNA1, DCC, RARB, ESR, AIM1, and RIZ1; (b) a higher frequency and quantity of methylation was noted in HNSCC compared with controls: TGFBR2, TIMP3, CDH1, MINT1, RBM6, and CALCA; (c) a higher frequency of methylation was noted in HNSCC with no difference in methylation levels: RASSF1A, PGP9.5, HIC1, DAPK, and CCND2; and (d) a similar frequency of methylation was noted in both groups; however, a quantitative difference was noted: S100A2. Overall results are shown on Table 3. Fourteen genes were associated with HNSCC with local recurrences ranging from 2.9 to 38.1.

TABLE 3
Comparison of hypermethylation detection on tumor
DNA (HNSCC patients) and serum samples (controls)

| | | | | Sensitivity, | Specificity, |
|---|---|---|---|---|---|
| Gene | Tumor case (n) | Serum control (n) | P* | % (95% CI) | % (95% CI) |
| HIC1 | 45 | 373 | <0.0001 | 100 (93.6-100) | 92.5 (89.3-95.0) |
| PGP9.5 | 45 | 203 | <0.0001 | 91.1 (78.8-97.5) | 97.5 (94.4-99.2) |
| TGFBR2 | 42 | 134 | <0.0001 | 88.1 (74.4-96.0) | 94.0 (88.6-97.4) |
| RARB | 44 | 85 | <0.0001 | 79.6 (64.7-90.2) | 100 (96.5-100) |
| DCC | 135 | 135 | <0.0001 | 77.8 (69.8-84.5) | 100 (97.8-100) |
| TIMP3 | 138 | 296 | <0.0001 | 73.9 (65.8-81.0) | 94.6 (91.4-96.9) |
| CCNA1 | 35 | 284 | <0.0001 | 68.6 (51.6-71.2) | 98.2 (97.2-100) |
| CDH1 | 77 | 320 | <0.0001 | 90.9 (82.2-96.3) | 73.1 (67.9-77.9) |
| CCND2 | 35 | 284 | <0.0001 | 68.6 (50.7-83.2) | 98.2 (95.9-99.4) |
| ESR | 35 | 20 | <0.0001 | 60.0 (42.1-76.1) | 100 (86.1-100) |
| MINT1 | 87 | 19 | <0.0001 | 90.8 (82.7-96.0) | 68.4 (43.5-87.4) |
| MINT31 | 136 | 42 | <0.0001 | 36.8 (28.7-45.5) | 100 (93.1-100) |
| AIM1 | 77 | 41 | <0.0001 | 23.4 (14.5-34.4) | 100 (93.0-100) |
| p16 | 136 | 102 | <0.0001 | 13.2 (8.0-20.1) | 100 (97.1-100) |
| CALCA | 35 | 20 | 0.005 | 100 (91.8-100) | 25.0 (8.7-49.1) |
| RASSF1A | 44 | 104 | 0.009 | 11.4 (3.8-24.6) | 99.0 (94.8-100) |
| RBM6 | 44 | 18 | 0.022 | 97.7 (88.0-100) | 22.2 (6.4-47.6) |
| S100A2 | 44 | 12 | 0.198 | 95.5 (84.5-99.4) | 16.7 (2.1-48.4) |
| RIZ1 | 44 | 18 | 0.550 | 6.8 (1.4-18.7) | 100 (84.7-100) |

*Fisher's exact test for the association between gene methylation and HNSCC.

### Initial Screening: Serum (Case) Versus Serum (Control).

Based on the above results, the genes that could distinguish the tumor samples (case) from serum samples (control) using binary results (presence or absence of methylation) and AUC>0.60 and at least 90% specificity or sensitivity were selected to be tested on serum from the HNSCC patients. Despite the fact that MINT1 and RBM6 would fulfill these criteria, the levels of methylation observed in the previous analysis showed higher levels of methylation in control serum than in tumor DNA, so these two markers were excluded as candidates for the panel. CCNA1, DCC, TIMP3, MINT31, p16, PGP9.5, AIM1, ESR, CCND2, CDH1, TGFBR2, and HIC1 were selected. It was noted, again, that despite the fact that some genes were quite specific for HNSCC compared with DNA from serum (controls), the sensitivity of these genes to be detected in the serum from the HNSCC patients was quite low or undetectable (Table 4A). Six genes were found to be significantly associated with HNSCC.

### HNSCC Detection in Serum in an Expanded Cohort.

Based on the above results, genes that could better distinguish the HNSCC patient salivary rinses from control salivary rinses, presenting an AUC>0.50 and at least 90% specificity and 10% sensitivity, were selected to be tested in combination for the expanded cohort. Based on these criteria, three genes were selected; however, a sufficient quantity of serum DNA allowed for evaluation of six genes. Therefore, it was decided to include CDH1 (due to its high sensitivity), CCND2, and TGFBR2. The results for AUC, sensitivity, and specificity for all possible combination with the selected genes are shown in FIG. 3), and the plot for sensitivity and specificity for these combinations is shown in FIG. 7B. The most favorable combinations are presented in Table 4B and show that some panels provide up to 84.5% specificity with 50.0% sensitivity and that specificity was observed as high as 92.5% in one combination of genes; however, sensitivity for this combination was only 31.4%. For other marker combinations, sensitivity was observed as high as 81.0%; however, the corresponding specificity was 43.5%.

TABLE 4
Comparison of hypermethylation detection for single genes on
serum samples (HNSCC patients) an serum samples (controls)
A. Individual gene evaluation

| | | | Sensitivity, | Specificity, |
|---|---|---|---|---|
| Gene | Case (n) | Control (n) | % (95% CI) | % (95% CI) |
| HIC1 | 70 | 373 | 31.4 (20.9-43.6) | 92.5 (89.3-95.0) |
| PGP9.5 | 52 | 203 | 7.7 (2.1-18.5) | 97.5 (94.4-99.2) |
| CDH1 | 62 | 320 | 32.3 (20.9-45.3) | 73.1 (67.9-77.9) |
| CCND2 | 47 | 284 | 6.4 (1.3-17.5) | 98.2 (95.9-99.4) |
| TIMP3 | 50 | 296 | 10.0 (3.3-21.8) | 94.6 (91.4-96.9) |
| TGFBR2 | 37 | 134 | 8.1 (1.7-21.9) | 94.0 (88.6-97.4) |
| AIM1 | 10 | 41 | 10.0 (0.3-44.5) | 100 (93.0-100) |
| ESR | 16 | 20 | 6.3 (0.2-30.2) | 100 (86.1-100) |
| CCNA1 | 24 | 104 | 0 (0-11.7) | 100 (97.2-100) |
| DCC | 27 | 135 | 0 (0-10.5) | 100 (97.8-100) |
| MINT31 | 28 | 42 | 0 (0-10.2) | 100 (93.1-100) |
| p16 | 39 | 102 | 0 (0-7.4) | 100 (97.1-100) |
| RARB | 13 | 85 | 0 (0-20.6) | 100 (96.5-100) |

B. Best combination of genes for hypermethylation HNSCC detection on serum

| | | | Sensitivity, | Specificity, |
|---|---|---|---|---|
| Panel | Case (n) | Control (n) | % (95% CI) | % (95% CI) |
| CCND2 + TIMP3 + HIC1 + PGP9.5 | 40 | 182 | 65.0 (48.3-79.4) | 72.0 (64.9-78.4) |
| TIMP3 + HIC1 | 52 | 278 | 50.0 (35.8-64.2) | 84.5 (79.7-88.6) |
| CCND2 + HIC1 + PGP9.5 | 42 | 189 | 52.4 (36.4-68.0) | 81.0 (74.6-86.3) |
| CCND2 + HIC1 | 49 | 248 | 44.9 (30.7-59.8) | 87.1 (82.3-91.0) |
| TIMP3 + HIC1 + PGP9.5 | 45 | 183 | 57.8 (42.2-72.3) | 74.3 (67.4-80.5) |
| CCND2 + TIMP3 + HIC1 | 46 | 178 | 56.5 (41.1-71.1) | 73.6 (66.5-79.9) |
| CCND2 + TGFBR2 + TIMP3 + HIC1 + PGP9.5 | 36 | 130 | 72.2 (54.8-85.8) | 56.9 (48.0-65.6) |
| CDH1 + CCND2 + TIMP3 + HIC1 + PGP9.5 | 39 | 208 | 87.2 (72.6-95.7) | 42.3 (35.5-49.3) |
| TGFBR2 + TIMP3 + HIC1 | 43 | 158 | 60.5 (44.4-75.0) | 68.4 (60.5-75.5) |
| TGFBR2 + TIMP3 + HIC1 + PGP9.5 | 38 | 131 | 68.4 (51.4-82.5) | 58.8 (49.9-67.3) |
| TGFBR2 + HIC1 | 44 | 149 | 50.0 (34.6-65.4) | 76.5 (68.9-83.1) |
| CDH1 + TIMP3 + HIC1 | 49 | 267 | 69.4 (54.6-81.8) | 57.3 (51.1-63.3) |
| CDH1 + CCND2 + HIC1 + PGP9.5 | 39 | 217 | 76.9 (60.7-88.9) | 49.3 (42.5-56.2) |
| HIC1 | 70 | 373 | 31.4 (20.9-43.6) | 92.5 (89.3-95.0) |
| TGFBR2 + HIC1 + PGP9.5 | 39 | 118 | 56.4 (39.6-72.2) | 66.9 (57.7-75.3) |
| CCND2 + TGFBR2 + TIMP3 + HIC1 | 40 | 126 | 65.0 (48.3-79.4) | 58.7 (49.6-67.4) |
| CCND2 + TGFBR2 + HIC1 + PGP9.5 | 37 | 117 | 59.5 (42.1-75.3) | 65.0 (55.6-73.6) |
| CDH1 + TIMP3 + HIC1 + PGP9.5 | 42 | 209 | 81.0 (65.9-91.4) | 43.5 (36.7-50.6) |
| HIC1 + PGP9.5 | 57 | 202 | 38.6 (26.0-52.4) | 84.2 (78.4-88.9) |

### Compartment-Specific Hypermethylation.

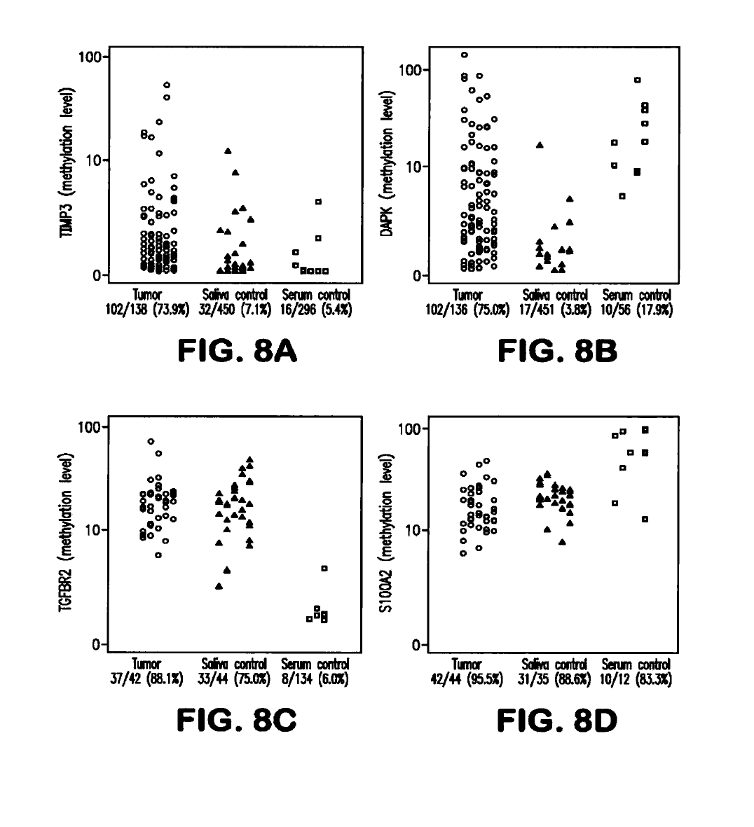

It was noted that some markers exhibited significant presence in normal control subject sera and salivary rinses, although sometimes in only one compartment (FIG. 8). For example, TIMP3 showed good specificity in distinguishing tumor samples from salivary rinses and serum control samples based on methylation results, allowing its use on both panels; on the other hand, S100A2 did not show any specificity for distinguishing groups based on salivary rinses and serum analysis. Some particular genes, such as TGFBR2, showed significant promoter methylation in salivary rinses from normal controls as well as tumor tissue from HNSCC patients but still showed a low frequency of methylation in sera from normal controls. This allows the use of TGFBR2 in a serum detection panel but prevents its use in detection of HNSCC in salivary rinses. Conversely, DAPK showed methylation in serum from normal control patients as well as in primary HNSCC, consistent with previous findings (Reddy et al., Cancer Res 2003; 63:7694-8), but only had minimal methylation in salivary rinses from control subjects, allowing for use in a salivary rinse detection panel. Other markers showed this phenomenon of compartment-specific methylation in normal controls (FIG. 8), and this phenomenon made a significant effect in the determination of separately constructed detection panels depending on the body fluid or cellular compartment of interest for detection.

Aberrant promoter hypermethylation has been proposed as a means for detection of tumor-specific cells in body fluids and exfoliated cells in solid tumors, including HNSCC. A large sample size of both controls and HNSCC patients using an expanded panel of methylated promoter regions to determine the ability of Q-MSP to detect tumor specific promoter methylation in serum and salivary rinses was studied. Salivary rinses obtained from rinses and brushing as a normal control tissue to obtain a broad representation of epithelial cells from the upper aerodigestive tract were used. Use of site matched control tissues, for example, would ignore the possibility of site-specific contamination from other sites in the upper aerodigestive tract (e.g., lymphoepithelial contamination in the oropharynx) and was therefore not used. Given the sensitivity of the Q-MSP technique used to detect the presence of methylated alleles in a background of normal at a threshold of 1/1,000 to 1/10,000, this strategy allowed methylated genes that were highly specific for tumor and rarely or never present in any of the aerodigestive sites that shed cells in salivary rinses to be defined. In this sense, the selection of a control tissue that was obtained in a manner similar to those that will likely be used in a surveillance or screening strategy provides an advantage in selecting appropriate targets in this analysis.

The significant role of promoter methylation as a means of epigenetic alteration in HNSCC based on the determination that ˜100% of the cases have shown methylation in the tumor DNA for at least one of the studied genes was confirmed. This would indicate the potential for use of aberrant promoter hypermethylation as a tool for detection of HNSCC and reinforce the potential for use of this technology in screening and surveillance. It was also confirmed that detection of tumor-specific promoter hypermethylation is feasible in body fluids and the Q-MSP is well adapted into a high-throughput format.

Some studies with limited cohorts have shown that promoter methylation was HNSCC specific and could not be detected in healthy controls in salivary rinse, mucosal cytobrush, or serum. An elevated frequency of promoter hypermethylation in HNSCC in a panel of gene promoters previously described as methylated in HNSCC as well other solid tumors was confirmed in this example. However, it was also found that normal control tissue showed substantial rates of methylation in a subset of these promoters. This would suggest that prior studies simply missed the phenomenon of promoter methylation in tissues from subjects without a cancer diagnosis due to small sample size. In addition, this observation could be explained by the phenomenon of compartment-specific methylation as a normal physiologic state. For example, RARB is hypermethylated in normal control salivary rinses at a similar frequency and magnitude when compared with primary HNSCC. Similar phenomena have been noted with other genes. Finally, promoter hypermethylation can be associated with age, race, or tobacco and alcohol exposure.

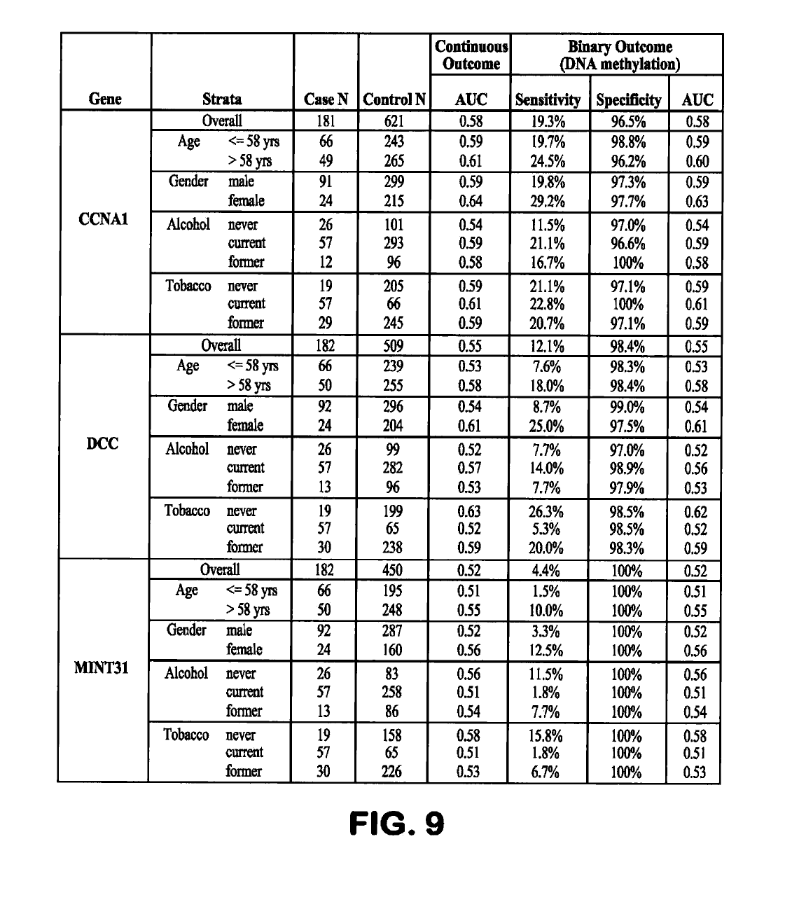

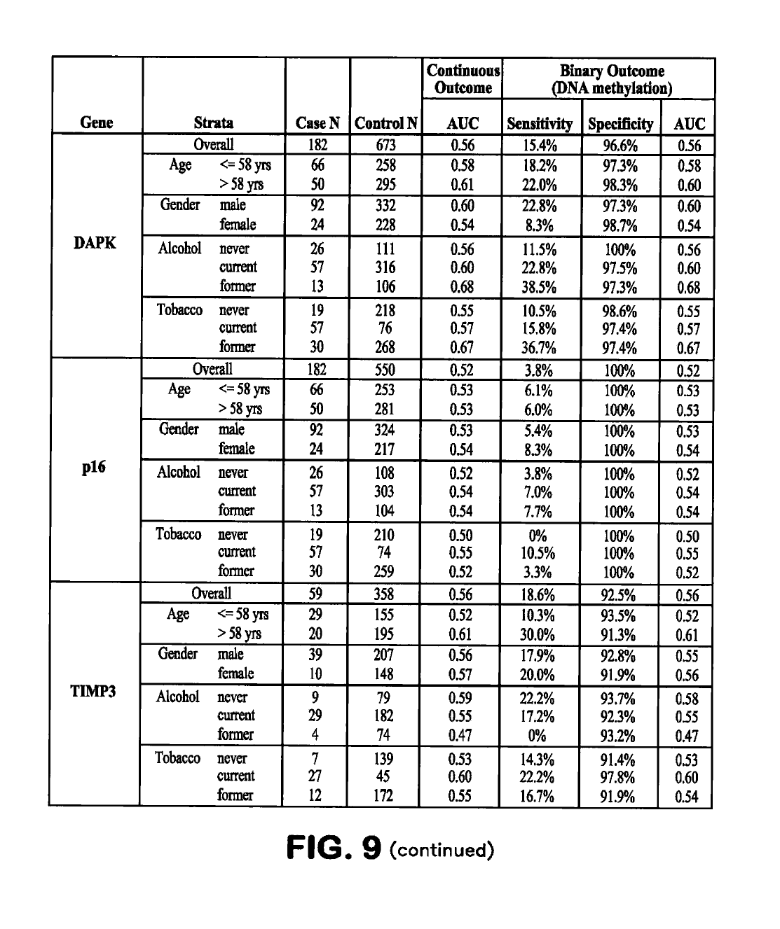

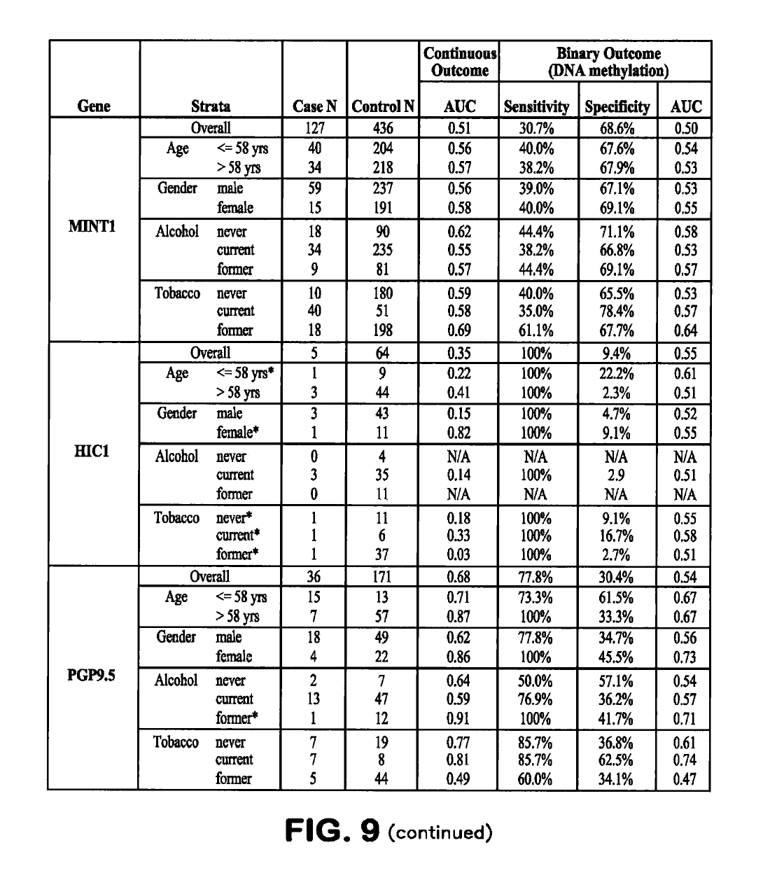

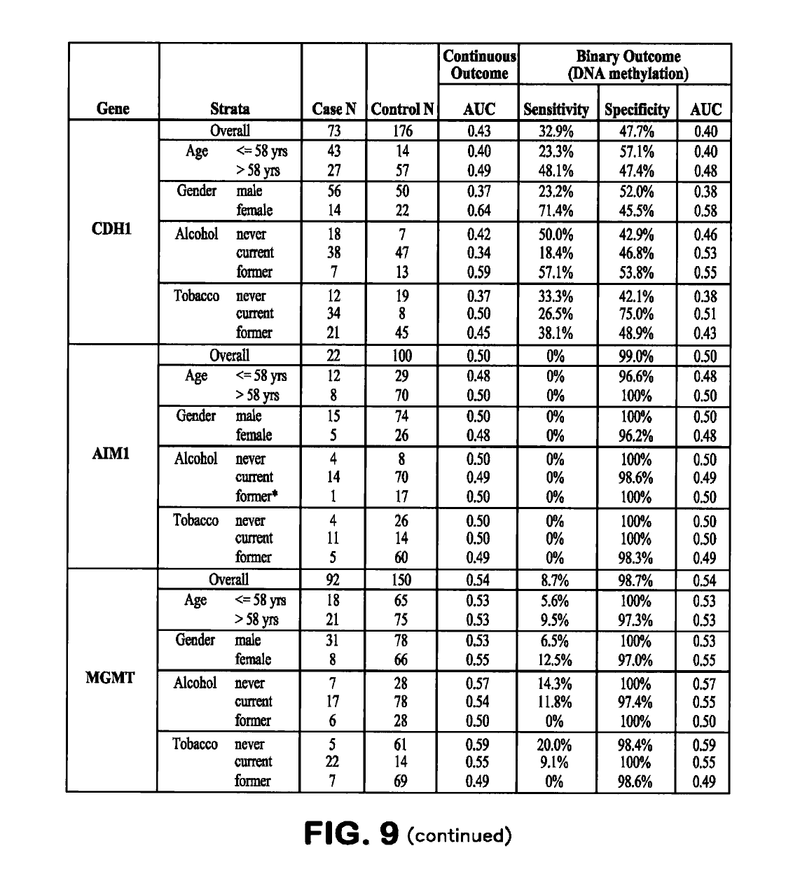

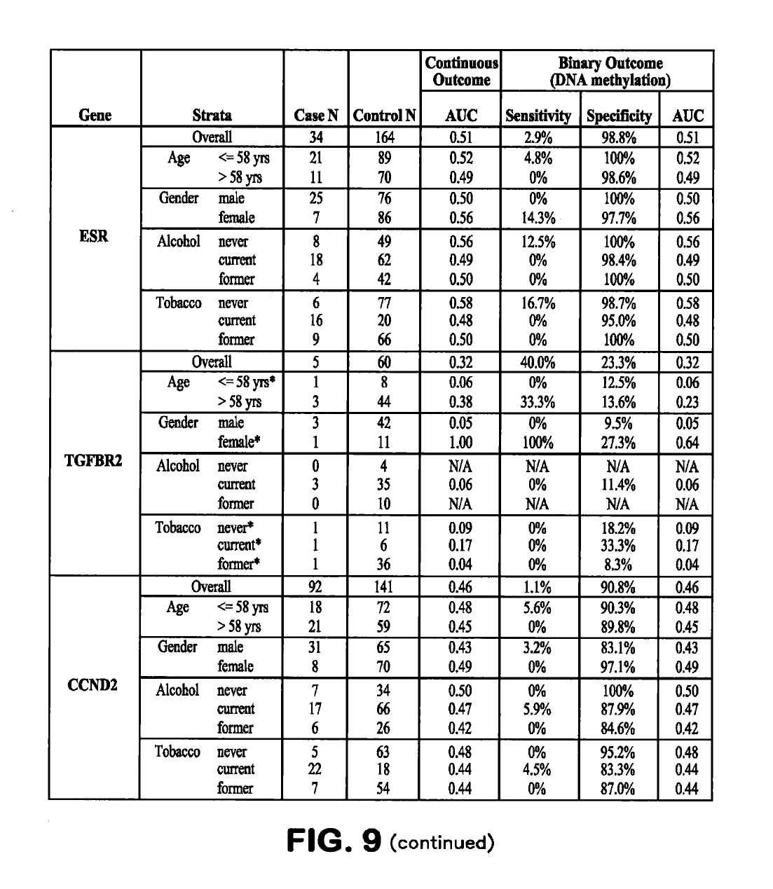

These effects may be combined in that studies showing very high specificity for hypermethylated genes in solid tumors often will use a few controls that may be biased to include young, nonsmoker, nondrinker controls, contributing toward a selection bias that artificially increases the false-negative frequency of promoter hypermethylation in controls. Often studies do not include control samples but only determine frequency of promoter hypermethylation in HNSCC primary tumor, salivary rinses, or serum from patients. It is important to notice that the control population in the current study can be considered as high risk for HNSCC; the majority of them were male, with median age ˜60 years and reported regular consumption of tobacco and alcohol. Due to concerns about age, gender, and tobacco or alcohol consumption as being described as associated to methylation, one additional analysis based on salivary rinse samples from cases and controls was performed. The results included the frequency distributions AUC, sensitivity, and specificity for each gene, which was summarized using both continuous and dichotomous methylation status (See FIG. 9). On this analysis, it was observed that the genes that were most specific for distinguishing cases and controls did not significantly change the AUC based on age, gender, and tobacco or alcohol consumption (CCNA1, DAPK, DCC, MGMT, TIMP3, MINT31, p16, AIM1, ESR, and CCND2). On the other hand, genes such as HIC1, TGFBR2, PGP9.5, MINT1, and CDH1 showed an important variance on AUC results depending on those factors. These results reinforce the observation that genes that are able to discriminate cases from controls in salivary rinse assays were not significantly influenced by age, gender, and tobacco or alcohol consumption. Finally, the use of Q-MSP may have increased the sensitivity in detecting low quantity methylation even in a subset of healthy controls. However, it was noted that the use of Q-MSP as a continuous variable did not show significant improvement compared with analysis as a binary variable.

The results demonstrated that the presence of promoter hypermethylation for selected genes proved to be highly specific for HNSCC in primary tumors (e.g., p16 and MINT31; Table 1). However, promoter hypermethylation is not always detectable in salivary rinses or in the serum from HNSCC patients despite the presence of methylation in primary tumors (Table 2A). This may be due to dilution effect of normal, nonmethylated genomes present in salivary rinses from normal mucosa.

It was also noted that there was significant variation in the shedding of tumor-specific methylated DNA into the serum compartment. For example, hypermethylated DNA was often not detected in the serum despite the fact that primary tumors were hypermethylated for that specific target. One possible explanation for this is that very low levels of methylation in a small minority of tumors was detected and that this would reduce the net amount of methylated DNA found in serum (e.g., DCC, CCNA1, and MINT31). On the other hand, some genes were found to be methylated in normal mucosa at low levels but would show a higher quantity of methylation in primary HNSCC and were useful as markers for detection in circulating serum. For example, the use of Q-MSP allowed discrimination between elevated levels of promoter methylation in serum when comparing HNSCC patients with normal controls despite similar rates of promoter methylation (HIC1 and PGP9.5). Finally, lack of detection in serum may be due to differential shedding of tumor DNA into the serum compartment that is tumor specific, also noted in other studies.

It was shown that whereas the use of single genes for detection is possible, using a combination of genes in a panel provides improvement in sensitivity. As promoter hypermethylation patterns in individual tumors show variation depending on specific altered molecular pathways, the use of multiple genes will provide greater applicability and coverage for diverse tumors when compared with a single gene for general detection. From the initial screening of 21 genes for salivary rinses, ultimately 8 genes were selected as part of a panel to distinguish salivary rinses from HNSCC patients and healthy controls (Table 2B). A combination of three or four genes was able to provide a sensitivity ranging from 24.0% to 35.1% with a specificity ranging from 90.0% to 97.1%.

From the six selected genes based on preliminary analysis for use in detection in serum, only HIC1 would be useful as a single gene marker, with a sensitivity of 31.4% and a specificity of 92.5%. Multiple gene combinations would add a much higher sensitivity (>65%) but would also be associated with a much lower specificity (<60%). However, other gene combinations could have sensitivity of 50.0% with a specificity of 84.5% (Table 4B). Prior reports using promoter hypermethylation to detect HNSCC in serum have shown an ˜35% correlation of primary tumor methylation with matched serum for most studies,. In the present study, the overall sensitivity in detecting the HNSCC in serum ranged from 31.4% to 87.2%, with the necessary caveat that improved sensitivity was obtained at the cost of decreased specificity. The relatively low sensitivity of serum-based detection using promoter hypermethylation has been described for other solid tumors as well.

In general, this study indicates that adequate assessment of the utility of promoter hypermethylation in HNSCC includes quantitative measurement of promoter methylation as well as a significant sized cohort of appropriately matched normal. In addition, the presence of promoter hypermethylation of tumor suppressor genes in control populations can happen as a random and perhaps even physiologic event. This methylation may be tissue specific and may also be related to age or environmental carcinogenic exposures. These factors significantly affect the selection of a control group, as limited size, young, healthy control group with minimal tobacco and ethanol exposure can bias reporting of falsely elevated specificity for candidate genes.

For the top 21 combinations of markers to detect HNSCC in salivary rinses, 3 of the combinations that had the lowest number of cases available for analysis included 150 cases and 240 controls. The other top 18 combinations had at least 174 cases and 416 controls (Table 2B). For the serum panel, the lowest number of cases available for one of the top marker panels was 36 cases and 130 controls, but other combinations included 70 cases and 373 controls (Table 4B).

In general, a panel for HNSCC detection with a high specificity but accompanied by a low sensitivity was defined. This combination of characteristics may not be as advantageous for population-based screening without improved sensitivity. However, panels with high sensitivity and low specificity were defined, which may have potential use for surveillance after a HNSCC treatment or surveillance in a high-risk population. Recent studies using the technology of CpG island microarray may be of use in helping to create a panel with higher sensitivity keeping the high specificity.

The findings of this experiment have also shown that the addition of novel detection markers in this context will focus on markers with high specificity, as the effect of addition of markers to a preexisting panel can only degrade specificity. Fortunately, there are some markers (p16 and DCC) that exhibit fairly high specificity, and additional markers with similar, highly specific characteristics are likely to be discovered or characterized in future studies.

### Example 2

### Surveillance HNSCC with Promoter Hypermethylation in Saliva

Hypermethylation of tumor suppressor gene promoters has been found in head and neck squamous carcinoma and other solid tumors. The present experiment evaluated these alteration in pre-treatment saliva from HNSCC patients using real-time quantitative MSP (Q-MSP).

Pretreatment saliva DNA samples from HNSCC patients were evaluated for patterns of hypermethylation using Q-MSP. Target tumor suppressor gene promoter regions were selected based on the results of Example 1 describing a screening panel for HNSCC in high risk population subjects. The selected genes were: DAPK, DCC, MINT-31, TIMP-3, p16, MGMT, CCNA1.

The experiment analyzed the panel in a cohort of 62 HNSCC patients. Thirty-three of the analyzed patients (53.2%) demonstrated methylation of at least one of the selected genes in the saliva DNA. Pre-treatment methylated saliva DNA was not significantly associated with tumor site (p=0.20) nor clinical stage (p=0.34). However, local disease control and overall survival were significantly lower in patients presenting hypermethylation in saliva rinses (p=0.01 and p=0.04, respectively). The multivariate analysis confirmed that this hypermethylation pattern remained as an independent prognostic factor for local recurrence (HR 5.77; 95% CI 1.2-27.0; p=0.02).

Example 2 discloses a panel for detection of HNSCC evaluating the saliva of the patients from Example 1. The present example confirmed an elevated rate of promoter hypermethylation ins HSNCC patients' saliva using a panel of gene promoters previously described in Example 1. The present example also determined that detection of hypermethylation in pre-treatment saliva DNA may be predictive of local recurrence. This finding may improve or influence treatment and surveillance of HNSCC patients.

### Materials and Methods

### Tissue Samples.

Samples were obtained from HNSCC patients presenting with a previously untreated squamous cell carcinoma from the oral cavity, larynx or pharynx. Patients were evaluated and enrolled in a protocol in the Department of Otolaryngology-Head and Neck Surgery at Johns Hopkins Medical Institutions, Baltimore Salivary rinse samples from these patients were collected prior to any cancer treatment.

Salivary rinses were obtained by brushing the oral cavity and oropharyngeal surfaces with an exfoliating brush followed by rinse and gargle with 20 ml normal saline solution. Cellular material from the brushing was released into the saline rinse, and centrifuged to obtain a cell pellet after supernatant was discarded.

The experimental protocol was approved by the Johns Hopkins Medical Institutions Institutional Review Board and informed consent was obtained from all enrolled subjects.

### DNA Extraction.

DNA obtained from tumor, salivary rinses and serum samples was extracted by digestion with 50 μg/ml proteinase K (Boehringer, Mannheim, Germany) in the presence of 1% SDS at 48° C. overnight, followed by phenol/chloroform extraction and ethanol precipitation.

### Patients and Methods

### Bisulfite Treatment.

DNA from tissue samples was subjected to bisulfite treatment, as described previously (Herman et al. PNAS 1996; 93:9821-6). Briefly, 2 μg of genomic DNA was denatured in 0.2 M of NaOH for 20 minutes at 50° C. The denatured DNA was diluted in 500 ill of freshly prepared solution of 10 mmol/l hydroquinone and 3M of sodium bisulfite and incubated for 3 hours at 70° C. After incubation, the DNA sample was desalted through a column (Wizard DNA Clean-Up System; Promega, Madison, Wis.), treated with 0.3 M of NaOH for 10 minutes at room temperature, and precipitated overnight with ethanol. The bisulfite-modified genomic DNA was resuspended in 120 μl of H₂O and stored at −80° C.

### Quantitative Methylation Specific PCR (Q-MSP).

The bisulfite-modified DNA was used as a template for fluorescence-based real-time polymerase chain reaction (PCR), as previously described Harden et al. Clin Cancer Res 2003; 9:1370-5. In brief, primers and probes were designed to specifically amplify the bisulfite-converted DNA for the ACTB gene and all genes of interest (primers and probes sequences are shown in FIG. 1). The ratios between the values of the gene of interest and the internal reference gene (ACTB), was obtained by Taqman analysis taking into account the PCR efficiency. Results were used as a measure of the relative quantity of methylation in a particular sample (value for the gene of interest/value for the reference gene x 100). Fluorogenic PCR reactions were carried out in a reaction volume of 20 μl consisting of 600 nM of each primer; 200 μM of probe; 0.75 U of platinum Taq polymerase (Invitrogen); 200 μM of each dATP, dCTP, dGTP, and dTTP; 200 nM of ROX Dye reference (Invitrogen); 16.6 mmol/1 of ammonium sulfate; 67 mmol/1 of Trizma (Sigma); 6.7 mmol/1 of magnesium chloride; 10 mmol/1 of mercaptoethanol; and 0.1% dimethylsulfoxide. Three microliters of treated DNA solution were used in each real-time MSP reaction. Amplifications were carried out in 384-well plates in a 7900 Sequence Detector System (Perkin-Elmer Applied Biosystems, Norwalk, Conn.). Thermal cycling was initiated with a first denaturation step at 95° C. for 2 min., followed by 45 cycles at 95° C. for 15 sec and 60 or 62° C. for 1 min. Leukocytes from a healthy individual were methylated in vitro with excess SssI methyltransferase (New England Biolabs) to generate completely methylated DNA, and serial dilutions of this DNA were used for constructing the calibration curves on each plate. Each reaction was performed in triplicate, the average of the triplicate was considered for analysis. Results for Q-MSP was analyzed considering the quantity of methylation (normalized by ACTB) as well as considering methylation as a binary event, in which any quantity of methylation in a sample would be considered as positive.

### Target Gene Selection.

Genes selected for this study came from a study previously demonstrated in Example 1 done to develop a panel for HNSCC detection in body fluids. The genes able to detect HNSCC in saliva rinse, and included in this example were: DAPK, DCC, MINT-31, TIMP-3, p16, MGMT, Cyclin-A1.

### Statistical Analysis.

Hypermethylation of each gene was treated as a binary variable (methylation vs. no methylation) by dichotomizing each gene at zero.

All analyses were performed using SPSS software (version 15.0). Descriptive analysis was performed to show the distribution of the population and the statistical comparisons using the chi-square test.

The survival analysis was done using the Kaplan-Meier method and the log-rank test. The disease free survival interval was defined as the interval between the date of the initial treatment and the recurrence. The local control time was defined as the interval between the date of initial treatment and diagnosis of local recurrence. The Cox proportional risk model was used to calculate the multifactorial risk of local recurrence or death. Statistical significance was determined for two-sided p-values <0.05.

### Results

Sixty-two patients were included in this study. HNSCC patients were mainly males (82.3%), caucasians (72.6%) with ages ranging from 32 to 84 years old (median, 58.3 years). Alcohol or tobacco consumption (current or past) were reported by 73.1% and 85.0%, respectively.

Primary tumor sites were: oral cavity, 30 cases (48.4%); oropharynx, 20 (32.3%) and larynx/hypopharynx, 12 (19.4%). Clinical stage at diagnosis was early in 12 cases (19.4%) (I and II); and advanced in 49 cases (79.0%) (clinical stage III and IV). In 1 case, information for staging was not available. All patients underwent surgical resection, and in 38 (61.3%) postoperative radiotherapy was done.

Promoter hypermethylation pattern of the 7 selected genes by tested in the primary tumor and pre-treatment saliva. Sixty-nine primary tumors (96.8%) had hypermethylation of at least one gene of the panel. In the pre-treatment salivary rinse, thirty-three patients (53.2%) presented with hypermethylation of at least one gene from the panel. Using selected combinations of the previously reported 10 gene panel for HNSCC detection, the hypermethylation detection rate in pre-treatment salivary rinses varied from 35.5% to 53.2% depending on the panel tested, being higher for the full panel.

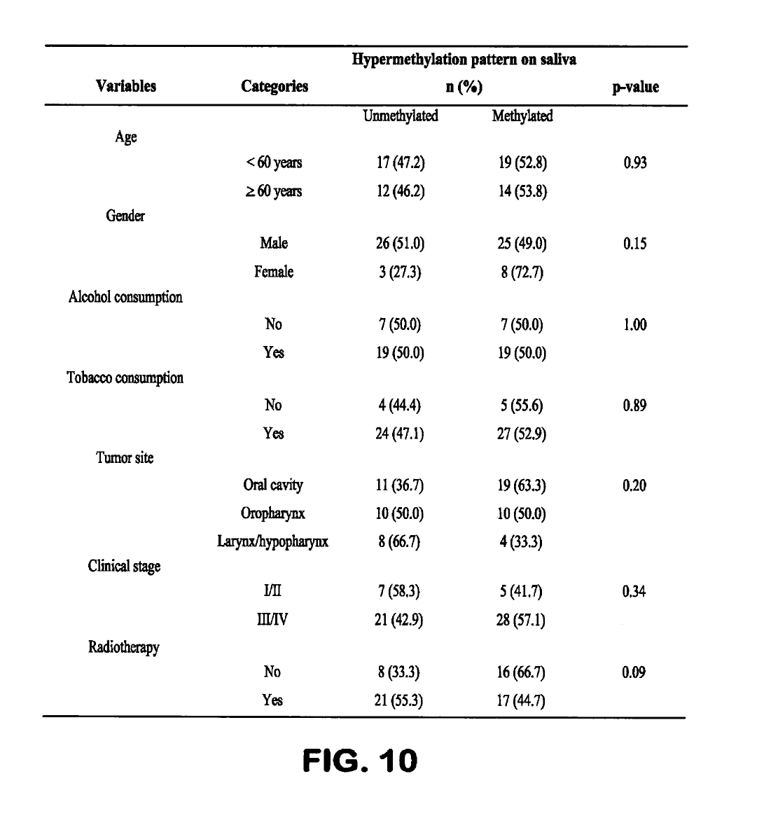

For this cohort of patients, detection of hypermethylation in pre-treatment saliva was not related to clinical variables as: age, gender, alcohol or tobacco consumption, tumor site or clinical stage (FIG. 10).

Recurrences occurred in 22 cases (35.5%), including local recurrence in 11 cases (17.7%); regional in 8 (12.9%) and distant in 8 (12.9%). Results include 5 patients with multi-site recurrences (8.1%). Local recurrence occurred in a median period of 15.7 months after initial treatment; with 81.8% of recurrences diagnosed prior to 2 years of follow-up.

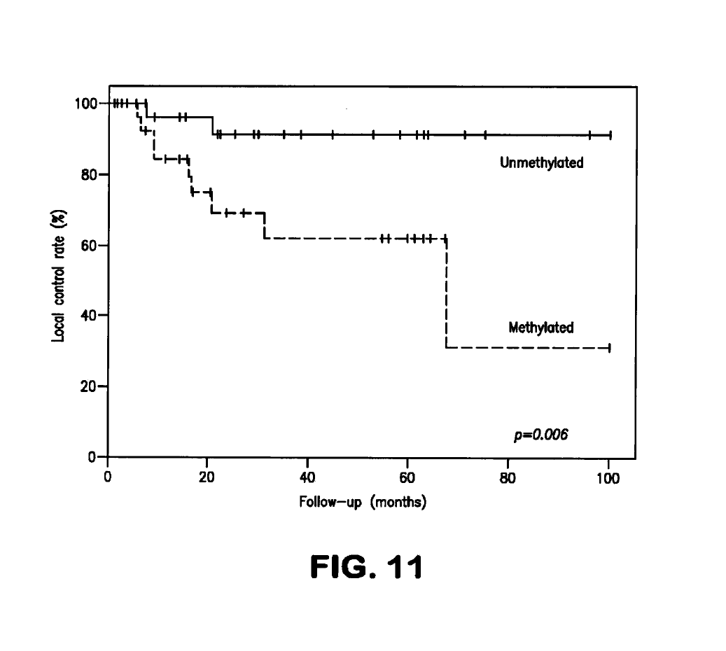

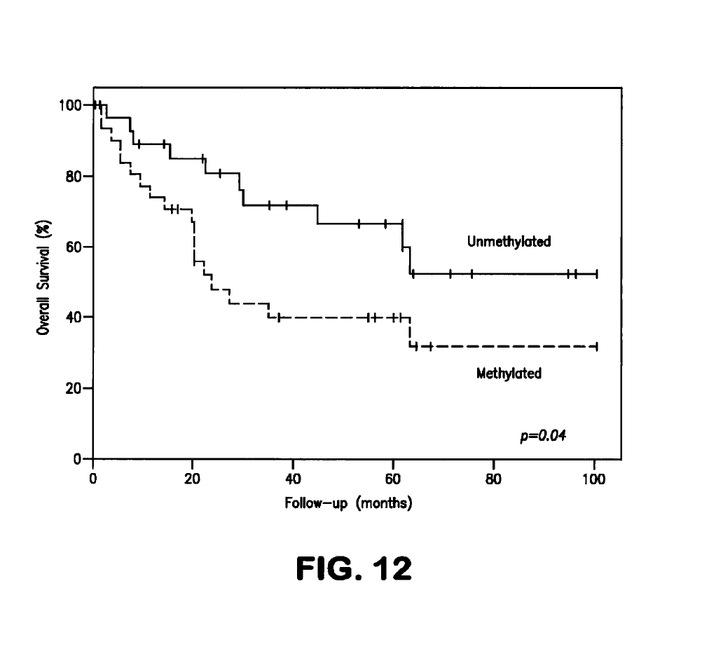

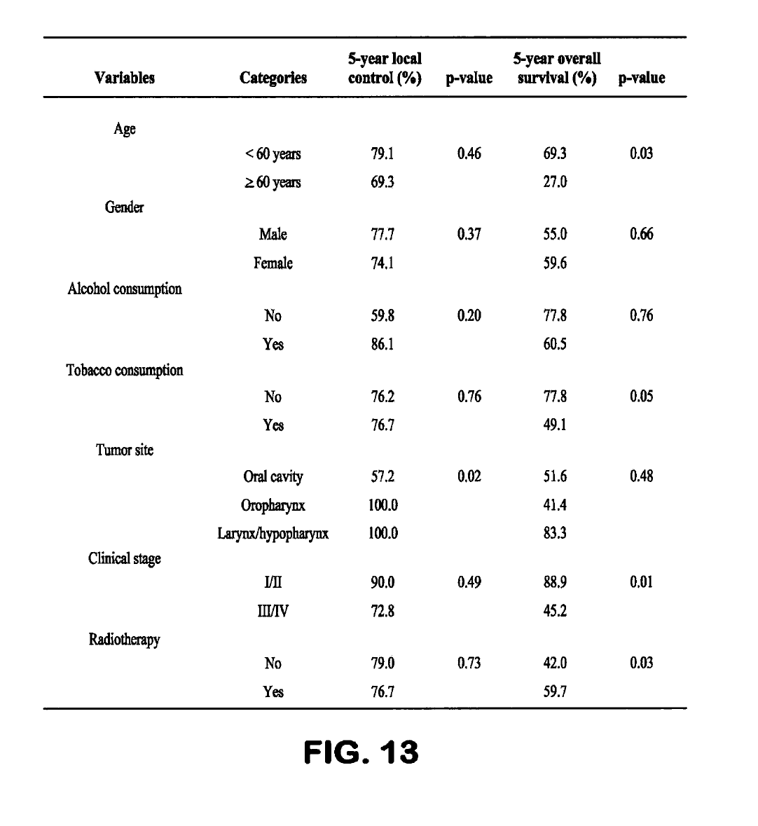

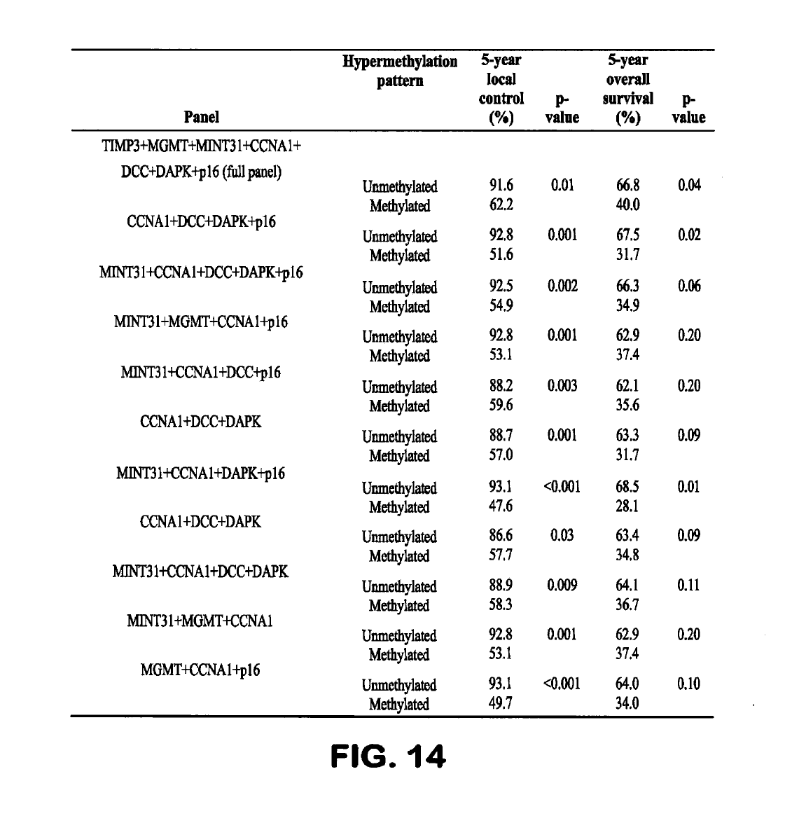

Local disease control rate at 5-years was 77.4%, varying from 62.2% for cases with hypermethylation detected in pre-treatment saliva rinses to 91.6% for the patients without hypermethylation (p=0.01) [FIG. 11]. Overall survival at 5-years was 52.9%, varying from 40.0% for cases with pre-treatment saliva rinse hypermethylation to 66.8% for cases without pre-treatment salivary rinse methylation (p=0.04) [FIG. 12]. Pre-treatment methylated saliva DNA was not significantly associated with tumor site (p=0.20) nor clinical stage (p=0.34) or with other clinical variables (FIG. 10). Tumor site was related to local control (p=0.01); regarding overall survival, age (p=0.03); tobacco consumption (p=0.05); clinical stage (p=0.01) and postoperative radiotherapy (p=0.03) were variables found to be related with prognosis (FIG. 13). Using the top 10 combinations from the previously reported gene panel, it was found that all tested combinations demonstrated statistically significant associations of pre-treatment salivary rinse methylation with poorer local control; yet three of them were found to be significantly associated with poorer overall survival (FIG. 14).

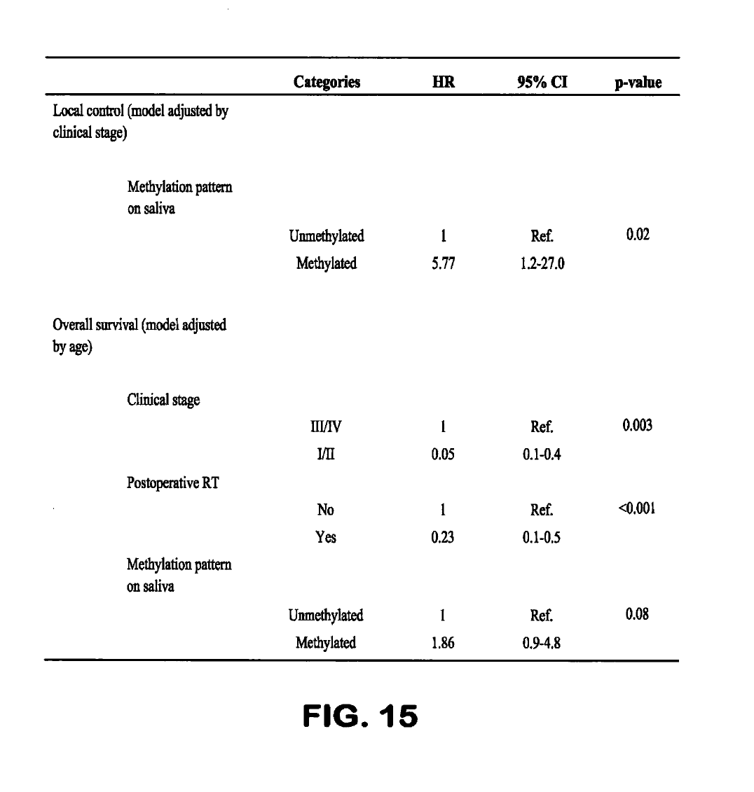

Multivariate analysis model for analysis of detection of hypermethylation in pre-treatment saliva rinses remained as an independent prognostic factor for local recurrence (HR 5.77; 95% CI 1.2-27.0; p=0.02) (FIG. 15).

Aberrant promoter hypermethylation has been proposed as a means for detection of tumor specific cells in body fluids and exfoliated cells in solid tumors, including HNSCC. In Example 1 a large sample size of both controls and HNSCC patients was evaluated using an expanded panel of methylated promoter regions to determine the ability of Q-MSP to detect tumor specific promoter methylation in serum and salivary rinses. This example used salivary samples obtained from rinses and brushing healthy individuals as normal control tissue in order to obtain a broad representation of epithelial cells from the upper aerodigestive tract. Given the sensitivity of the Q-MSP technique used to detect the presence of methylated alleles in a background of normal at a threshold of 1/1,000 to 1/10,000, this strategy allowed the inventors to define methylated genes that were highly specific for tumor, and rarely or never present in any of the aerodigestive sites that shed cells in salivary rinses. From the initial screening of 21 genes for salivary rinses, ultimately seven genes were selected as part of a panel to distinguish salivary rinses from HNSCC patients and healthy controls. A combination of 3 or 4 genes is able to provide a sensitivity of cancer detection ranging from 24.0% to 35.1% with a specificity ranging from 90.0% to 97.1% Carvalho et al. Sites of recurrence in oral and oropharyngeal cancers according to the treatment approach. Oral Dis 2003; 9:112-8).

Those findings confirmed that detection of tumor specific promoter hypermethylation is feasible in body fluids and the Q-MSP is well adapted into a high throughput format.

In general, the results defined a panel for HNSCC detection with a high specificity but accompanied by a low sensitivity. However, experimental results enabled the definition of panels with high sensitivity and low specificity, which have potential use for surveillance after treatment or in a high risk population. It was decided to test the hypothesis that pretreatment salivary rinses may be associated with clinical outcome, and evaluate the utility of our panel in predicting local recurrence in HNSCC patients.

Righini et al. Clin Cancer Res 2007; 13:1179-85 evaluated a cohort of 90 patients for the utility of methylation detection in saliva pre and post-treatment, among the 22 patients suitable for follow-up. Hypermethylation on post-operative salivary rinses were analyzed, including 6 patients with recurrence. Among those, 5 patients demonstrated hypermethylation in postoperative salivary rinses, only 1 case without recurrence showed methylation in saliva.

In the present study, the detection of hypermethylation in pre-treatment salivary rinses was significantly related to local control and overall survival. Interestingly, hypermethylated HNSCC salivary rinses were not associated with tumor site or clinical stage, and were noted to be an independent risk factor for local control and overall survival in the multivariate analysis.

The prognostic significance of hypermethylation in pretreatment salivary rinses is related to a higher concentration of methylated signal in exfoliated cells, independent of tumor stage or site, and therefore is unlikely to be related to tumor volume per se. However, there are multiple, possibly complementary explanations for this association. Aggressive tumors with poorer prognosis may undergo increased rate of mechanical dissociation or shedding into salivary rinses. Those tumors with a higher burden of epigenetic alteration would be more frequently detected in salivary rinses, and may have a more aggressive behavior. Other explanations include the phenomenon of lateral clonal expansion, in which premalignant clonal patches expand well beyond primary tumor location, resulting in a larger surface area of epigenetically altered cells to shed into the saliva, and also may predispose to development of recurrent tumors from adjacent premalignant cells.

This example illustrates that the above experiments were able to confirm an elevated rate of promoter hypermethylation detected in HNSCC patients saliva using a panel of gene promoters previously described as methylated in HNSCC but not in control subjects. In addition, detection of hypermethylation in pre-treatment saliva DNA is associated with local recurrence. This has implication for further study regarding the mechanism of this observation, but also may have practical applications for increasing intensity of surveillance, or using adjunctive therapy for local control in patients with promoter hypermethylation in pretreatment salivary rinses.

### Example 3

Silencing of tumor suppressor genes plays a role in head and neck carcinogenesis. Methylation of CpG islands in the promoter region of genes acts as a significant mechanism of epigenetic gene silencing. In this example the aim was to evaluate the epigenetic changes specific to head and neck squamous cell carcinoma (HNSCC) by investigating aberrant promoter hypermethylation of a panel of four genes (EDNRB, p16, DCC and KIF1A) via candidate gene approaches.

### Materials and Methods

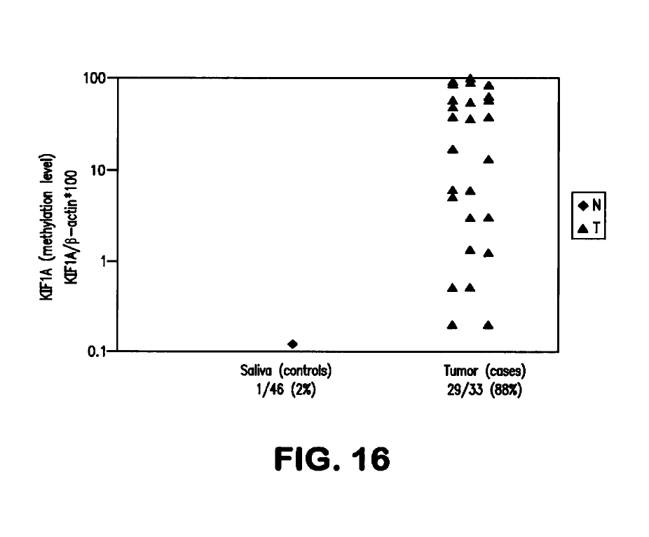

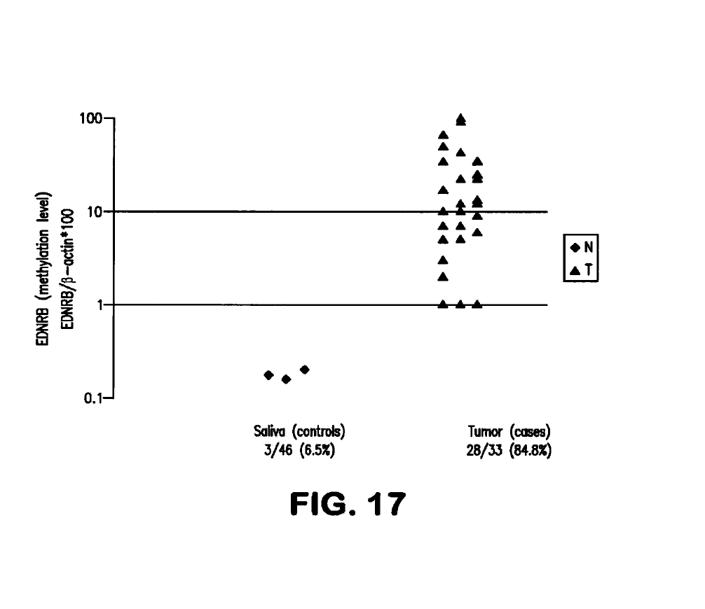

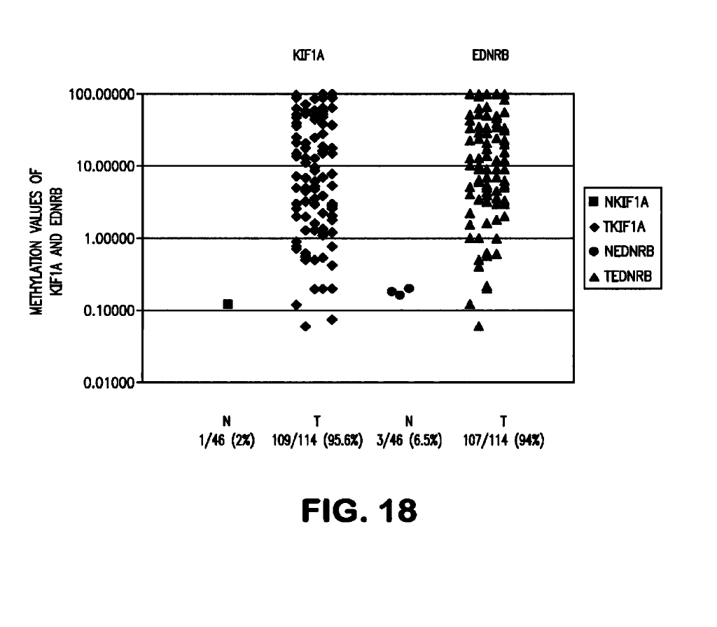

In this study the investigation of the methylation of the gene promoters by bisulfite modification and quantitative methylation-specific PCR (Q-MSP) that provides more objective and rapid estimation of gene methylation status, was performed in a preliminary study of a limited cohort of normal saliva samples (n=46) and patients with HNSCC (n=33) (FIGS. 16 and 17). In a further study, the methylation status of two selected genes (EDNRB, KIF1A) were analyzed in 114 patients with HNSCC (FIG. 18).

KIF1A and EDNRB demonstrated minimal (2% and 6.5%, respectively) methylation in normal salivary rinses, but were found to be highly methylated (95.6% and 94%, respectively) in primary HNSCC.

Methylation of the KIF1A and EDNRB gene promoters is a frequent event in HNSCC and these genes are not methylated in normal salivary rinses, demonstrating potential for these genes as biomarkers in detection strategies.

Primers for EDNRB may include for RT-methylation specific PCR:

Forward Primer
(SEQ ID NO: 67)
5′-GGTTACGCGGGGGAAGAAAAATAGTTG-3′,
Taqman Probe
(SEQ ID NO: 68)
5′-CATAACTCGCCAACGCGAATCGAAACTCC-3′,
Reverse Primer
(SEQ ID NO: 69)
5′-ATACCGCCCGCAACCTCTTCG-3′

(See also Yegnasubramanian et al., Cancer Res. 64:1975-1986, 2004; Hogue et al., Cancer Res. 68:2661-2670, 2008)

Although the invention has been described with reference to the above examples, it will be understood that modifications and variations are encompassed within the spirit and scope of the invention. Accordingly, the invention is limited only by the following claims.

What is claimed is:

1. A method for diagnosing a subject having or at risk of developing head and neck cancer comprising:
determining the methylation state of a gene or the regulatory region of at least two genes in a nucleic acid sample from the subject, wherein the at least two genes or regulatory regions are hypermethylated as compared to the same regions in a corresponding normal cell;
wherein the regulatory regions of the at least one of the two genes is selected from the group consisting of DCC, DAPK, TIMP3, ESR, CCNA1, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, EDNRB and a combination thereof.

2. The method of claim 1, wherein at least two regulatory regions in two genes are identified as hypermethylated.

3. The method of claim 1, wherein the head and neck cancer is head and neck squamous cell carcinoma (HNSCC).

4. The method of claim 1 wherein sample is selected from the group consisting of a saliva and serum sample.

5. The method according to claim 4, wherein the combination of genes includes at least one gene selected from the group consisting of CCNA1, TIMP3, DCC, DAPK, MGMT, MINT31, p16, PGP9.5, MINT1, CDH1, AIM1, ESR, CCND2 and a combination thereof.

6. The method of claim 1 wherein the combination of genes comprises a panel of from about two to twenty-five genes or regulatory regions thereof.

7. The method according to claim 4, wherein the combination of genes includes one gene selected from the group consisting of HIC1, PGP9.5, CDH1, CCND2, TIMP3, TGFBR2, AIM1, ESR, CCNA1, DCC, MINT31, p16, RARB and a combination thereof.

8. The method of claim 1 wherein the hypermethylation is at a CpG dinucleotide motif in the at least one gene or regulatory region.

9. The method of claim 8, wherein the hypermethylation is determined using quantitative methylation-specific PCR (Q-MSP).

10. A method of determining the prognosis of a subject having a head and neck cancer comprising:
determining the methylation state of a gene or the regulatory region of at least two genes in a nucleic acid sample from the subject, wherein the at least two genes or regulatory regions are hypermethylated as compared to the same regions in a corresponding normal cell;
wherein the regulatory regions of at least one of the two genes is selected from the group consisting of DCC, DAPK, TIMP3, ESR, CCNA1, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, and EDNRB and a combination thereof;
and wherein the hypermethylation of the region as compared to the same region in a corresponding normal cell is indicative of a poor prognosis.

11. The method of claim 10, wherein at least two regulatory regions in two genes are identified as hypermethylated.

12. The method of claim 10 wherein sample is selected from the group consisting of a saliva and serum sample.

13. The method according to claim 10, wherein the combination of genes includes at least one gene selected from the group consisting of CCNA1, TIMP3, DCC, DAPK, MGMT, MINT31, p16, PGP9.5, MINT1, CDH1, AIM1, ESR, CCND2 and a combination thereof.

14. The method according to claim 10, wherein the combination of genes includes at least one gene selected from the group consisting of HIC1, PGP9.5, CDH1, CCND2, TIMP3, TGFBR2, AIM1, ESR, CCNA1, DCC, MINT31, p16, RARB and a combination thereof.

15. A method for determining whether a subject is responsive to a particular therapeutic regimen comprising:
determining the methylation state of a gene or the regulatory region of at least two genes, in a nucleic acid sample from the subject, wherein the at least two genes or regulatory regions are hypermethylated as compared to the same regions in a corresponding normal cell;
wherein the regulatory regions of at least one of the two genes is selected from the group consisting of DCC, DAPK, TIMP3, ESR, CCNA1, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, EDNRB and a combination thereof;
wherein the hypermethylation of the region as compared to the same region in a corresponding normal cell is indicative of a subject who may be responsive to the therapeutic regimen.

16. The method of claim 14, wherein the therapeutic regimen is administration of a chemotherapeutic agent.

17. The method of claim 16, wherein the chemotherapeutic agent is selected from the group consisting of methotrexate, cisplatin/carboplatin, canbusil, dactinomicin, taxol (paclitaxol), a vinca alkaloid, a mitomycin-type antibiotic, a bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine, vindesine, tamoxifen, and mechlorethamine.

18. The method of claim 14, wherein the therapeutic regimen is administration of a demethylating agent.

19. The method of claim 18, wherein the agent is 5-azacytidine, 5-aza-2-deoxycytidine or zebularine.

20. A kit comprising:
an agent that provides a determination of the methylation state of a gene or the regulatory region of a gene; and
a panel of at least two genes and/or regulatory regions of the genes wherein at least one gene is selected from the group consisting of DCC, DAPK, TIMP3, ESR, CCNA1A, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, EDNRB and a combination thereof.

21. A kit comprising:
an agent that provides a determination of the methylation state of a gene or the regulatory region of a gene; and
a panel of at least two genes and/or regulatory regions of the genes wherein at least two genes are selected from the group consisting of DCC, DAPK, TIMP3, ESR, CCNA1A, CCND2, MINT1, MINT31, CDH1, AIM1, MGMT, p16, PGP9.5, RARB, HIC1, RASSF1A, CALCA, TGFBR2, S100A2, RIZ1, RBM6, KIF1, EDNRB and a combination thereof.

22. The kit of claim 20 wherein the gene or regulatory region thereof is determined with cells of the subject from a saliva sample.

23. The kit according to claim 22, wherein the combination of genes includes at least CCNA1A, TIMP3, DCC, DAPK, MGMT, MINT31, p16, PGP9.5, MINT1, CDH1, AIM1, ESR, CCND2 and a combination thereof.

24. The kit of claim 21 wherein the gene or regulatory region thereof is determined with cells of the subject from a serum sample.

25. The kit according to claim 24, wherein the combination of genes includes at least HIC1, PGP9.5, CDH1, CCND2, TIMP3, TGFBR2, AIM1, ESR, CCNA1, DCC, MINT31, p16, RARB and a combination thereof.

26. The kit of claim 21 wherein the gene or regulatory region thereof is determined with cells of the subject from a saliva sample.

27. The kit according to claim 26, wherein the combination of genes includes at least CCNA1A, TIMP3, DCC, DAPK, MGMT, MINT31, p16, PGP9.5, MINT1, CDH1, AIM1, ESR, CCND2 and a combination thereof.


SEQUENCE LISTING (69 sequences)

SEQ ID NO: 1 - DNA, 25 bp, Artificial Sequence
  Primer
tggtgatggaggaggtttagtaagt

SEQ ID NO: 2 - DNA, 30 bp, Artificial Sequence
  Probe
accaccacccaacacacaataacaaacaca

SEQ ID NO: 3 - DNA, 27 bp, Artificial Sequence
  Primer
aaccaataaaacctactcctcccttaa

SEQ ID NO: 4 - DNA, 20 bp, Artificial Sequence
  Primer
cgcgggtattggatgttagt

SEQ ID NO: 5 - DNA, 24 bp, Artificial Sequence
  Probe
gggagcgttgcggattattcgtag

SEQ ID NO: 6 - DNA, 20 bp, Artificial Sequence
  Primer
ccgacccacctatacgaaaa

SEQ ID NO: 7 - DNA, 23 bp, Artificial Sequence
  Primer
gttttggaagtatgagggtgacg

SEQ ID NO: 8 - DNA, 30 bp, Artificial Sequence
  Probe
attccgccaatacacaacaaccaataaacg

SEQ ID NO: 9 - DNA, 19 bp, Artificial Sequence
  Primer
ttcccgccgctataaatcg

SEQ ID NO: 10 - DNA, 26 bp, Artificial Sequence
  Primer
aattttaggttagagggttatcgcgt

SEQ ID NO: 11 - DNA, 19 bp, Artificial Sequence
  Probe
cgcccacccgacctcgcat

SEQ ID NO: 12 - DNA, 22 bp, Artificial Sequence
  Primer
tccccaaaacgaaactaacgac

SEQ ID NO: 13 - DNA, 18 bp, Artificial Sequence
  Primer
tcgcggcgagtttattcg

SEQ ID NO: 14 - DNA, 22 bp, Artificial Sequence
  Probe
cgttatggcgatgcggtttcgg

SEQ ID NO: 15 - DNA, 16 bp, Artificial Sequence
  Primer
ccgaccgcgacaaacg

SEQ ID NO: 16 - DNA, 27 bp, Artificial Sequence
  Primer
tttgatttaaggatgcgttagagtacg

SEQ ID NO: 17 - DNA, 24 bp, Artificial Sequence
  Probe
aatccgccaacacgatcgacccta

SEQ ID NO: 18 - DNA, 25 bp, Artificial Sequence
  Primer
actttctccctaaaaaccgactacg

SEQ ID NO: 19 - DNA, 24 bp, Artificial Sequence
  Primer
ggatagtcggatcgagttaacgtc

SEQ ID NO: 20 - DNA, 28 bp, Artificial Sequence
  Probe
ttcggtaattcgtagcggtagggtttgg

SEQ ID NO: 21 - DNA, 16 bp, Artificial Sequence
  Primer
ccctcccaaacgccga

SEQ ID NO: 22 - DNA, 21 bp, Artificial Sequence
  Primer
ttgttcgcgatttttggtttc

SEQ ID NO: 23 - DNA, 25 bp, Artificial Sequence
  Probe
gcgctaaacaaaaaaactccgaaaa

SEQ ID NO: 24 - DNA, 22 bp, Artificial Sequence
  Primer
accgattacttaaaaatacgcg

SEQ ID NO: 25 - DNA, 19 bp, Artificial Sequence
  Primer
ggcgttcgttttgggattg

SEQ ID NO: 26 - DNA, 24 bp, Artificial Sequence
  Probe
cgataaaaccgaacgacccgacga

SEQ ID NO: 27 - DNA, 19 bp, Artificial Sequence
  Primer
gccgacacgcgaactctaa

SEQ ID NO: 28 - DNA, 19 bp, Artificial Sequence
  Primer
gttaggcggttagggcgtc

SEQ ID NO: 29 - DNA, 31 bp, Artificial Sequence
  Probe
caacatcgtctacccaacacactctcctacg

SEQ ID NO: 30 - DNA, 19 bp, Artificial Sequence
  Primer
ccgggcgcctccatcgtgt

SEQ ID NO: 31 - DNA, 23 bp, Artificial Sequence
  Primer
cgaatatactaaaacaacccgcg

SEQ ID NO: 32 - DNA, 26 bp, Artificial Sequence
  Probe
aatcctcgcgatacgcaccgtttacg

SEQ ID NO: 33 - DNA, 21 bp, Artificial Sequence
  Primer
gtattttttcgggagcgaggc

SEQ ID NO: 34 - DNA, 22 bp, Artificial Sequence
  Primer
attttcgaagcgtttgtttggc

SEQ ID NO: 35 - DNA, 23 bp, Artificial Sequence
  Probe
gcgaaactcccctactctccaac

SEQ ID NO: 36 - DNA, 20 bp, Artificial Sequence
  Primer
acaaaaaacctcaaccccgc

SEQ ID NO: 37 - DNA, 22 bp, Artificial Sequence
  Primer
gagtgatttattaggtttcgtc

SEQ ID NO: 38 - DNA, 23 bp, Artificial Sequence
  Probe
acgccgaaaaacacttccccaac

SEQ ID NO: 39 - DNA, 19 bp, Artificial Sequence
  Primer
cgaaaacgaaacgccgcga

SEQ ID NO: 40 - DNA, 24 bp, Artificial Sequence
  Primer
ttattagagggtggggcggatcgc

SEQ ID NO: 41 - DNA, 23 bp, Artificial Sequence
  Probe
agtagtatggagtcggcggcggg

SEQ ID NO: 42 - DNA, 21 bp, Artificial Sequence
  Primer
gaccccgaaccgcgaccgtaa

SEQ ID NO: 43 - DNA, 22 bp, Artificial Sequence
  Primer
cggcgagtgagattgtaaggtt

SEQ ID NO: 44 - DNA, 28 bp, Artificial Sequence
  Probe
ttcggtcgtattatttcgcgttgcgtac

SEQ ID NO: 45 - DNA, 24 bp, Artificial Sequence
  Primer
gaacgatcgcgaccaaataaatac

SEQ ID NO: 46 - DNA, 27 bp, Artificial Sequence
  Primer
gggattagaattttttatgcgagttgt

SEQ ID NO: 47 - DNA, 22 bp, Artificial Sequence
  Probe
tgtcgagaacgcgagcgattcg

SEQ ID NO: 48 - DNA, 20 bp, Artificial Sequence
  Primer
taccccgacgatacccaaac

SEQ ID NO: 49 - DNA, 18 bp, Artificial Sequence
  Primer
gcgttgaagtcggggttc

SEQ ID NO: 50 - DNA, 24 bp, Artificial Sequence
  Probe
acaaacgcgaaccgaacgaaacca

SEQ ID NO: 51 - DNA, 24 bp, Artificial Sequence
  Primer
cccgtacttcgctaactttaaacg

SEQ ID NO: 52 - DNA, 20 bp, Artificial Sequence
  Primer
ggtttattcgtcgcggttta

SEQ ID NO: 53 - DNA, 25 bp, Artificial Sequence
  Probe
taactcgaaacctccttattatccg

SEQ ID NO: 54 - DNA, 20 bp, Artificial Sequence
  Primer
aactcgaatacgaccctaac

SEQ ID NO: 55 - DNA, 20 bp, Artificial Sequence
  Primer
ggattcgcggtgatttacga

SEQ ID NO: 56 - DNA, 23 bp, Artificial Sequence
  Probe
cgacggcgtagggttaagggtcg

SEQ ID NO: 57 - DNA, 26 bp, Artificial Sequence
  Primer
ctacgaaactaaaaaactccgaaacc

SEQ ID NO: 58 - DNA, 22 bp, Artificial Sequence
  Primer
tggtttcgattttttgatttcg

SEQ ID NO: 59 - DNA, 26 bp, Artificial Sequence
  Probe
cgaccgaacgcgataacttactccta

SEQ ID NO: 60 - DNA, 25 bp, Artificial Sequence
  Primer
tcaaaattctttttacaacaacgcc

SEQ ID NO: 61 - DNA, 17 bp, Artificial Sequence
  Primer
gaggggaggcggtagat

SEQ ID NO: 62 - DNA, 22 bp, Artificial Sequence
  Probe
cgacgtccaacccctaactctc

SEQ ID NO: 63 - DNA, 21 bp, Artificial Sequence
  Primer
caacttcaactcaacgctacg

SEQ ID NO: 64 - DNA, 21 bp, Artificial Sequence
  Primer
gcgtcggaggttaaggttgtt

SEQ ID NO: 65 - DNA, 19 bp, Artificial Sequence
  Probe
aactcgctcgcccgccgaa

SEQ ID NO: 66 - DNA, 22 bp, Artificial Sequence
  Primer
ctctccaaaattaccgtacgcg

SEQ ID NO: 67 - DNA, 27 bp, Artificial Sequence
  Primer
ggttacgcgggggaagaaaaatagttg

SEQ ID NO: 68 - DNA, 29 bp, Artificial Sequence
  Probe
cataactcgccaacgcgaatcgaaactcc

SEQ ID NO: 69 - DNA, 21 bp, Artificial Sequence
  Primer
ataccgcccgcaacctcttcg